(19) United States
(12) Patent Application Publication
(10) Pub. No.: US 2024/0052005 A1
(43) Pub. Date: Feb. 15, 2024

(54) IMPROVED CELL-PENETRATING PEPTIDES AND FUSION PROTEINS

(71) Applicant: ANASTASIS BIOTEC LIMITED, London (GB)

(72) Inventor: Agamemnon EPENETOS, London (GB)

(21) Appl. No.: 18/257,827

(22) PCT Filed: Dec. 16, 2021
(86) PCT No.: PCT/GB2021/053333

§ 371 Date: Jun. 15, 2023

(30) Foreign Application Priority Data
Dec. 16, 2020 (GB) 2019864.4

(51) Int. Cl. C07K 14/50; C12N 15/62

(52) U.S. Cl. CPC C07K 14/50; C12N 15/62; A61K 38/00

(57) ABSTRACT

The present invention relates to diagnostic and therapeutic molecules comprising derivatives of a cell-penetrating protein and any appropriate dominant-negative peptide and/or protein.

### FIELD OF THE INVENTION

The invention relates to the development of diagnostic and therapeutic molecules comprising derivatives of a cell-penetrating protein and any appropriate dominant-negative peptide and/or protein.

### BACKGROUND INFORMATION

The Notch pathway is one of the few highly conserved pathways that have different effects on the development and construction of various tissues. The canonical Notch pathway is initiated by ligands from either Jagged or Delta family. Several proteolytic activities are activated upon ligand binding, these activities result in intramembrane proteolysis of by the gamma-secretase complex and the release of the intracellular part of the Notch (ICN) receptor (De Strooper et al., 1999). Essential components of the activated Notch pathway transduction include ICN, the transcription factor CSL/RBPJ and transcriptional co-activators of the Mastermind-like (MAML) family (MAML1-3) (Jarriault et al., 1995; Tamura et al., 1995; Wu et al., 2000; Wu et al., 2002).

The bulk majority of the protein therapeutics fail or have low efficacy due to the fact that the therapeutic proteins cannot penetrate the cell membrane or have low potency of penetration inside cells, where the protein is the most effective. There is thus a need for more effective cell transducing agents which can deliver cargo molecules into cells, through tissues and across biological barriers such that the cargo can act in a therapeutic mechanism.

### SUMMARY OF THE INVENTION

The purpose of this invention is to create a new protein therapeutic that acts as a carrier and can transport a payload across a cell membrane. The therapeutic of the invention can thus deliver a pharmaceutically active substance inside a cell, where it can be most active and useful.

The present invention is based on the discovery that derivatives of a cell-penetrating peptide known as Antennapedia, for example, penetratin and variants thereof, has improved therapeutically-useful properties which leads to the creation of effective therapies of various diseases. The agent of the invention comprises three components: 1) a cell-penetrating peptide, 2) a dominant-negative functional effector protein, and 3) a peptide or chemical linker. In a preferred embodiment of the invention, the cell-penetrating peptide is derived from Antennapedia.

The present invention provides:
[1] A cell-penetrating peptide (CPP) comprising from 10 to 60 contiguous amino acids selected from an Antennapedia (ANTP) protein.
[2] The cell-penetrating peptide (CPP) according to [1], comprising from 10 to 60 contiguous amino acids selected from the sequence of SEQ ID NO: 2, or a variant thereof having at least 80% sequence identity to a sequence of from 10 to 60 contiguous amino acids selected from the sequence of SEQ ID NO: 2.
[3] The cell-penetrating peptide (CPP) according to [2], having the sequence of SEQ ID NO: 4 or a sequence having at least 80% sequence identity to SEQ ID NO: 4.
[4] The cell-penetrating peptide (CPP) according to [2] or [3], wherein the stability and/or transduction efficiency are improved compared to the stability and/or transduction efficiency of a CPP consisting of the sequence of SEQ ID NO: 2 and/or SEQ ID NO: 4.
[5] The cell-penetrating peptide (CPP) according to any one of [1] to [4], wherein the immunogenicity of the CPP is reduced compared to the immunogenicity of a CPP consisting of the sequence of SEQ ID NO: 2 and/or SEQ ID NO: 4.
[6] The cell-penetrating peptide (CPP) according to any one of the preceding aspects, wherein, compared to the sequence of SEQ ID NO: 2, the CPP variant comprises a mutation of any arginine to lysine or another tolerated residue.
[7] The cell-penetrating peptide (CPP) according to any one of the preceding aspects, wherein, compared to the sequence of SEQ ID NO: 2, the CPP variant comprises one or more alternative thiol residues at positions 1 to 60.
[8] The cell-penetrating peptide (CPP) according to any one of the preceding aspects, wherein, compared to the sequence of SEQ ID NO: 2, the CPP variant comprises mutation of one or more non-basic residue into a basic residue.
[9] The cell-penetrating peptide (CPP) according to any one of the preceding aspects, wherein, compared to the sequence of SEQ ID NO: 2, the CPP variant comprises the introduction of any stabilising mutations, such as disulphide bridges, ionic interactions, hydrophobic interactions, reduced proteolysis liabilities.
[10] The cell-penetrating peptide (CPP) according to any one of the preceding aspects, wherein, compared to the sequence of SEQ ID NO: 2, the CPP variant comprises chemical modification of any residue 1-60, such as alkylation, cross-linking, or stapling.
[11] The cell-penetrating peptide (CPP) according to any one of the preceding aspects, wherein, compared to the sequence of SEQ ID NO: 2, the CPP variant comprises the addition of any chemical modification at the N-terminus or C-terminus; optionally, wherein the modification improves the chemical conjugation, membrane translocation or stability properties of the CPP.
[12] The cell-penetrating peptide (CPP) according to any one of the preceding aspects, wherein, compared to the sequence of SEQ ID NO: 2, the CPP variant comprises the replacement of any natural amino acid (L-amino acid) with non-natural amino acids (D-amino acid).
[13] The cell-penetrating peptide (CPP) according to any one of the preceding aspects, wherein, the residue corresponding to Cys-39 in SEQ ID NO: 2 is replaced with any tolerated residue, such as Ser or Ala.
[14] The cell-penetrating peptide (CPP) according to [13], wherein the residue corresponding to Cys-39 in SEQ ID NO: 2 is replaced with a serine.
[15] The cell-penetrating peptide (CPP) according to [14] comprising SEQ ID NO: 16.
[16] The cell-penetrating peptide (CPP) according to any one of the preceding aspects, comprising an additional cysteine at its N- and/or C-terminus.
[17] The cell-penetrating peptide (CPP) according to any one of the preceding aspects comprising SEQ ID NO: 13, SEQ ID NO: 14 or SEQ ID NO:15.
[18] A fusion protein comprising the cell-penetrating peptide (CPP) as defined in any one of [1] to [17] and a therapeutically useful protein.
[19] The fusion protein according to [18] wherein the therapeutically useful protein is a dominant-negative protein; optionally wherein the dominant-negative protein is a dominant-negative mastermind-like protein (DN-MAML), AKT-DN or STAT3; further optionally wherein the DN-MAML has the sequence of SEQ ID NO: 9 or a sequence with 80% sequence identity thereto.
[20] The fusion protein according to [20], wherein the cell-penetrating peptide (CPP) is covalently linked to the dominant-negative protein by a zero-length crosslinker, a homobifunctional crosslinker, a hetero-bifunctional crosslinker or a trifunctional crosslinkers; optionally wherein
the zero-length crosslinker is selected from carbodiimides (EDC, EDC plus Sulfo-NHS, CMC, DCC, DIC), Woodward's Reagent K, N,N-Carbonyldiimidazole, Schiff Base Formation and Reductive Amination);
the homobifunctional crosslinkers is selected from Homobifunctional NHS Esters (DSP and DTSSP, DSS and BS3, DST and Sulfo-DST, BSOCOES and Sulfo-BSOCOES, EGS and Sulfo-EGS, DSG, DSC), Homobifunctional Imidoesters (DMA, DMP, DMS, DTBP), Homobifunctional Sulfhydryl-Reactive Crosslinkers (DPDPB, BMH), Difluorobenzene Derivatives (DFDNB, DFDNPS), Homobifunctional Photoreactive Crosslinkers (BASED), Homobifunctional Aldehydes (Formaldehyde, Glutaraldehyde), Bis-epoxides (1,4-Butanediol Diglycidyl Ether), Homobifunctional Hydrazides, Adipic Acid, Dihydrazide, Carbohydrazide), Bis-diazonium Derivatives (o-Tolidine, Diazotized, Bis-diazotized Benzidine), Bis-alkyl Halides;
the hetero-bifunctional Crosslinker is selected from Amine-Reactive and Sulfhydryl-Reactive Crosslinkers (SPDP, LC-SPDP, and Sulfo-LC-SPDP, SMPT and Sulfo-LC-SMPT, SMCC and Sulfo-SMCC, MBS and Sulfo-MBS, SIAB and Sulfo-SIAB, SMPB and Sulfo-SMPB, GMBS and Sulfo-GMBS, SIAX and SIAXX, SIAC and SIACX, NPIA), Carbonyl-Reactive and Sulfhydryl-Reactive Crosslinkers (MPBH, M2C2H, PDPH), Amine-Reactive and Photoreactive Crosslinkers (NHS-ASA, Sulfo-NHS-ASA, and Sulfo-NHS-LC-ASA, SASD, HSAB and Sulfo-HSAB, SANPAH and Sulfo-SANPAH, ANB-NOS, SAND, SADP and Sulfo-SADP, Sulfo-SAPB, SAED, Sulfo-SAMCA, p-Nitrophenyl Diazopyruvate, PNP-DTP), Sulfhydryl-Reactive and Photoreactive Crosslinkers (ASIB, APDP, Benzophenone-4-iodoacetamide, Benzophenone-4-maleimide), Carbonyl-Reactive and Photoreactive Crosslinkers (ABH), Carboxylate-Reactive and Photoreactive Crosslinkers (ASBA), Arginine-Reactive and Photoreactive Crosslinkers (APG); and
the trifunctional crosslinker is selected from 4-Azido-2-nitrophenylbiocytin-4-nitrophenyl ester, Sulfo-SBED, MTS-ATF-Biotin and MTS-ATF-LC-Biotin, Hydroxymethyl Phosphine Derivatives.
[21] The fusion protein according to any one of [18] to [20], wherein
the cell-penetrating peptide (CPP) is as defined in [16];
the therapeutically useful protein is DN-MAML; and
DN-MAML is conjugated to the cell penetrating peptide via a thioester bond formed with a cysteine thiol at the N or C terminus of the ANTP sequence;
   optionally wherein:
the DN-MAML protein comprises the sequence of SEQ ID NO: 9 or a sequence with 80% sequence identity thereto; and/or
the DN-MAML protein comprises a maleimide group.
[22] A pharmaceutical composition comprising the cell penetrating peptide as defined in any one of [1] to [17] or the fusion protein as defined in any one of [18] to [21].
[23] The cell penetrating peptide as defined in any one of [1] to [17] or the fusion protein as defined in any one of [18] to [21] or the pharmaceutical composition as defined in [22] for use in a method of therapy.
[24] The cell penetrating peptide as defined in any one of [1] to [17] or the fusion protein as defined in any one of [18] to [21] or the pharmaceutical composition as defined in [22] for use in method of treating cancer.
[25] The cell penetrating peptide, the fusion protein or the pharmaceutical composition for use as defined in [24], wherein administration of the cell penetrating peptide, fusion protein or the pharmaceutical composition inhibits Notch signalling in a cancer cell.
[26] A method of treating cancer, the method comprising the administration of a cell penetrating peptide as defined in any one of [1] to [17] or the fusion protein as defined in any one of [18] to [21] or the pharmaceutical composition as defined in [22] to a subject in need thereof.
[27] Use of the cell penetrating peptide as defined in any one of [1] to [17] or the fusion protein as defined in any one of [18] to [21] or the pharmaceutical composition as defined in [22] in the manufacture of a medicament for the treatment of cancer.
[28] A method for producing a fusion protein, the method comprising linking the cell-penetrating peptide (CPP) as defined in any one of [1] to [17] and a therapeutically useful protein by any of the following methods: activated-esters coupling of lysine residues; methanesulfonyl acrylate coupling of lysine residues; copper and non-catalysed ‘click’ reactions of alkynes introduced into proteins; disulphide bonds formed with native or engineered cysteine thiol groups; thioether bonds formed with cysteine thiols and introduced maleimide groups; native chemical ligation; smartag chemo-selective ligation using formyl-glycine modified proteins; chemoselective azo-coupling reactions; cyclo-addition reactions (Cu-AAC or SPAAC) of azide-derivatized amino acids; and/or photo-reactive or photo-catalysed chemical reactions.
[29] A method for producing an ANTP fusion protein, the method comprising modifying the ANTP sequence by site directed mutagenesis to (i) replace the residue corresponding to Cys-39 in SEQ ID NO: 2 with a serine, and (ii) add a cysteine to the N or C terminus of the ANTP sequence, and conjugating the modified ANTP sequence to a therapeutic protein using thiol-maleimide chemistry.
[30] The method of [29], wherein the therapeutic protein is DN-MAML or a derivative thereof.
[31] The method of [30], wherein the DN-MAML has the sequence of SEQ ID NO: 9 or a sequence with 80% sequence identity thereto.
[32] The method of [30] or [31], wherein the DN-MAML sequence has bene modified to introduce a maleimide group.
[33] The method of any one of [29] to [32], wherein the ANTP sequence comprises SEQ ID NO: 2 or a sequence having 80% sequence identity thereto

### BRIEF DESCRIPTION OF THE FIGURES

FIG. 1 Overview of the canonical Notch signalling pathway and various experimental pharmacological inhibitors under development

FIG. 2 Intranuclear trafficking of ANTP and ANTP-p21 conjugates into the nuclei of SKOV-3 tumour cells. The p21 on its own did not translocate to the nuclei.

FIG. 3 Expression and purification of ANTP

FIG. 4 CD spectra that shows that the purified ANTP is expressed as a folded, helical-rich protein.

FIG. 5 Production of low yields of recombinant ANTP-dnMAML:
a. Soluble (S) and Insoluble (I) fractions of recombinant ANTP-dnMAML expressed in E. coli BL21(DE3) using a T7-based expression vector.
b. Anti-HIS blotting to confirm TR4 identity
c. Refolding by stepwise dialysis, wherein S is the starting sample, 11 (Lane-1) is 6M urea dialysis, 12 is 4M urea dialysis, 13 is 2M urea dialysis, and 14 is the remaining soluble and folded TR4.

FIG. 6 Mammalian expression of recombinant ANTP-dnMAML conjugate is non-viable:
Anti-HIS Western blot of E. coli recombinant ANTP-dnMAML (prepared according to Example 1) and (Live) cell extract (no TR4 present) and (Debris) small amount of recombinant ANTP-dnMAML present after CHO transfected mammalian cell culture.

FIG. 7 In vivo efficacy studies showing that recombinant ANTP/DN-MAML is less potent than Syntana-4 in nude mouse xenograft models

FIG. 8 Synthesis and characterisation of Syntana-4 ‘pre-folded’ peptide

FIG. 9 CD Analyses of Syntana-4

FIG. 10 Concentration of Syntana-4

FIG. 11 3D structure of ANTP. Cysteine residue labelled with dashed arrow, lysines with bold arrow and arginine residues with arrows.
The structure was generated using data was from Qian et al. Proc Natl Acad Sci USA. 1994 Apr. 26; 91(9):4091-5 and Swiss PDB viewer.

FIG. 12 Commercially-available dyes used to conjugate onto Syntana-4

FIG. 13 Conjugated Syntana-4 fluorescence spectra
(A) Syntana-4-IR dye spectra
(B) Syntana-4-Cy5 dye spectra
(C) Syntana-4-Cy5.5 dye spectra.
(D) SDS PAGE gels

FIG. 14 Two examples of in vivo efficacy studies showing that Syntana-4 causes significant tumour growth delays in nude mouse xenograft models of MDA-MB-231 tumours

FIG. 15 RT-Quantitative-PCR
Upper panel shows the relative expression of mRNA for various NOTCH-related genes and the lower panel is a graphical representation.

FIG. 16 Immuno-histochemistry images of Syntana-4 treated tumours, staining for Ki67 proliferative marker

FIG. 17 Flow cytometry of MDA-MB-231 cells treated with Syntana-4 Apoptotic cells were identified and quantified by Annexin V-DAPI staining. Cells were plated at 15,000 cells/well and treated 48 h later in triplicate with Syntana-4, ANTP or doxorubicin. After 72 h, cells were analysed by Flow cytometry. Bottom left quadrant indicates live cells, bottom right indicates early apoptosis, top right indicates late apoptosis, top left indicates dead cells.
The proportion of apoptotic cells was calculated using the histograms.

FIG. 18 Cell proliferation inhibition of MDA-MB-231 cells treated with Syntana-4 *p<0.05, **p<0.01, ***p<0.001

FIG. 19 Schematic showing the mutagenesis of ANTP to produce variants. In particular, site directed mutagenesis was used to remove the internal cysteine (C39S) and to add either an N or C terminal cysteine.

FIG. 20 Schematic showing cys-maleimide conjugation of cargo moieties.

FIG. 21 FITC-Mal conjugation of wild type ANTP

### DETAILED DESCRIPTION

### Cell Penetrating Peptides (CPPs)

The first component of the agent of the invention comprises a cell penetrating peptide (CPP). CPPs are also known as protein transduction domains (PTDs), membrane transduction peptides (MTPs), and Trojan peptides. This class of proteins has 8-15 positively charged amino acids and possesses a unique ability to cross cellular membranes (Cronican et al., 2011). These proteins are a special type of vectors that have a unique set of advantages including high internalisation rates, low toxicity and potential for sequence modification. Typically, CPPs are composed of 30-60 amino acids, and they are either derived from proteins or are synthesised as biomolecule-internalising vectors (Zhang et al., 2016, Balhassani et al. 2011).

he origin of CPP can also vary. For example, a CPP and a fragment thereof can be chimeric and derive from two or more dissimilar peptides. Transportan, for example, is a chimeric CPP composed of galanin and mastoparan. A CPP can also be protein-derived (e.g. TAT and penetratin (the cell-penetrating domain of Antennapedia, ANTP)). The polyarginine family is the example of synthesised CPPs. The amino acid sequence of an amphipathic model peptide is KLALKLALKALKAALKLA (SEQ ID NO: 17)(Lindgren et al., 2000).

ANTP can successfully deliver tumour-suppressor protein p21 into the cancer cells, where its expression is depleted. The resulting therapeutic protein (AB1, previously known as TR1) is disclosed in U.S. Pat. No. 10,259,852 B2. This novel cancer treatment was based on the published evidence that in many cancers, the expression of tumour suppressors such as p21 and p53 that regulate the cell cycle is impaired, which causes uncontrolled cellular division.

Mechanisms, by which CPPs pass through the membrane can involve endocytosis or direct perturbations of the lipid bilayer of the cell membrane. Direct translocation is observed at a relatively high CPP concentration, and when the cargo size is relatively small.

Translocation via endocytosis is commonly observed when CPPs are joined with a larger molecule (Jones et al., 2012, Erazo-Oliveras et al., 2012).

The mechanisms of action, by which CPPs enter cellular membranes can be attributed to the presence of the positively charged amino acids starches that interact with acidic motifs in the plasma membranes in receptor-independent fashion. Acidic motifs can include different substrates on the plasma membrane, such as proteoglycans and glycolipids. After this interaction, CPPs undergo cell-type independent internalisation, the exact mechanisms of which are yet to be discovered. The internalisation of CPPs such as Antp and Tat can be mediated by clathrin-dependant endocytosis, caveolin-dependant endocytosis, micropinocytosis, or direct cellular translocation (Duchardt et al., 2007). Quite remarkably, after cellular internalisation CPP-tagged therapy (CTT) escapes lysosomal degradation. Moreover, lysosomal escape can be enhanced using specific CPP modifications. US 2014/0038281 discloses chemically modified CPP TP10 that has improved endosomal escape efficiency.

Once the CPP conjugated with a therapeutic molecule is released into the cytoplasm, it is able to interact with its target. The CPPs uptake rates are remarkably fast. Thus, an in vitro study of a CPP Tat-linked therapy showed to enter the cells and start interacting with the target five minutes after the exposure to the treatment (Wadia et al., 2004). In vivo experiments using mouse lines for cancer, also confirmed the efficacy of CTT, and that a CPP conjugate can reach the tumour site within 30 minutes of the treatment (Bitler et al., 2009).

Standard and well-established methods can be used to test and to demonstrate the ability of a naturally occurring or synthetic sequences to cross through the cellular membrane. Some modifications and variants of the domains of CPPs that retain the ability of the proteins to translocate the cellular membrane are reported and are included in the scope of the present invention. These modifications include but are not limited to the variants disclosed by G. Osman (2018) PEGylated enhanced cell-penetrating peptide nanoparticles for lung gene therapy.

In a preferred embodiment of the invention, the cell-penetrating peptide is derived from Antennapedia (ANTP). For example, penetratin is the major cell-penetrating helix of ANTP protein consisting of the first 16 amino acids derived from the third helix of the Antp protein homeodomain. Penetratin has a natural ability to enter and deliver cargo to the nerve cells (Rizutti et al., 2015, Vasconcelos et al., 2013). ANTP contains two additional alpha-helices which enhances its CPP properties, is highly conserved throughout vertebrate species, and it is able to penetrate virtually every cell type and can pass through the blood-brain barrier.

In one embodiment, the “first component” comprises or consists of a cell-penetrating peptide derived from ANTP. The amino acid sequence of naturally-occurring ANTP comprises the 60 amino acid sequence (SEQ ID NO:2) below:

| | |
|---|---|
| Position | 1 2 3 4 5 6 7 8 9 10 11 12 13 14 15 16 17 18 19 20 |
| | R K R G R Q T Y T R Y Q T L E L E K E F |
| Position: | 21 22 23 24 25 26 27 28 29 30 31 32 33 34 35 36 37 38 39 40 |
| | H F N R Y L T R R R R I E I A H A L C L |
| Position: | 41 42 43 44 45 46 47 48 49 50 51 52 53 54 55 56 57 58 59 60 |
| | T E R Q I K I W F Q N R R M K W K K E N |

Variant cell-penetrating peptides of the invention may be produced by the removal of one or more amino acids from the N and/or C-terminal ends of SEQ ID NO:2. Truncations may also be generated by one or more internal deletions. The truncated derivatives may comprise or essentially consist of one or more alpha helices (α1, α2, or α3). In one embodiment, the CPP moiety is a 16 amino acid truncation of ANTP known as penetratin (SEQ ID NO:4, below). These residues correspond to residues 43 to 58 of ANTP (i.e. α3 helix of the helix-loop-helix-turn-helix motif). Accordingly, in some embodiments, the cell-penetrating peptide comprises or consists essentially of penetratin or suitable variants thereof.

| | |
|---|---|
| Position: | 43 44 45 46 47 48 49 50 51 52 53 54 55 56 57 58 |
| | R Q I K I W F Q N R R M K W K K |

In one embodiment, the cell-penetrating peptide comprises a sequence of from 10 to 60 contiguous amino acids selected from SEQ ID NO: 2. For example, the cell-penetrating peptide may comprise at least 15, 20, 25, 30, 35, 40, 45, 50, 55, or 60 contiguous amino acids selected from SEQ ID NO:2. The sequence of contiguous amino acids may comprise the amino acid corresponding to position 1 of SEQ ID NO: 2, the amino acid corresponding to position 60 of SEQ ID NO: 2. In one embodiment, the cell-penetrating peptide comprises a sequence having at least 80%, 85%, 90%, 95%, or 100% sequence identity to SEQ ID NO:2. Sequence identity may be calculated across the whole length of SEQ ID NO:2 or across the length of the corresponding fragment.

In another embodiment, the cell-penetrating peptide comprises a sequence of at least 10, 11, 12, 13, 14, 15, or 16 contiguous amino acids selected from SEQ ID NO:4. The sequence of contiguous amino acids may be selected from the N or C terminus of SEQ ID NO:4. In one embodiment, the cell-penetrating peptide comprises a sequence having at least 80%, 85%, 90%, 95%, or 100% sequence identity to SEQ ID NO:4. Sequence identity may be calculated across the whole length of SEQ ID NO:2 or across the length of the corresponding fragment.

Sequence identity may be determined using one of a number of online programmes, including but not limited to ToPLign (BioSolveIT GmbH, Germany), BLAST2 (NCBI), SUPERMATCHER (L'InstitutPasteur, France), MATCHER (EMBOSS), or ClustalW (Thompson et al., 1994, supra). Parameters will be known to the skilled person.

Also described are variant cell-penetrating peptides comprising 1, 2, 3, 4, 5, 6, 7, 8, 9, 10, or up to 20 amino acid substitutions or deletions compared to SEQ ID NO:2. Alternatively, a variant cell-penetrating peptide may comprise 1, 2, 3, or 4 amino acid substitutions or deletions compared to penetratin (SEQ ID NO:4). Preferably, amino acid substitutions are conservative in nature. For example, an amino acid may be substituted with an alternative amino acid having similar properties, (i.e. another basic amino acid, another acidic amino acid, another neutral amino acid, another charged amino acid, another hydrophilic amino acid, another hydrophobic amino acid, another polar amino acid, another aromatic amino acid or another aliphatic amino acid). Properties of the 20 naturally occurring amino acids are summarised below. This table can be used by the skilled person to establish which amino acids and be substituted. For example, lysine (K) residues are polar, hydrophilic, and positively charged, and can therefore be replaced by Arg (R) residues.

TABLE 1
Exemplary conservative substitutions

| Original Residue | Exemplary Conservative Substitution |
|---|---|
| Ala | Val, Leu, Ile |
| Arg | Lys, Gln, Asn |
| Asn | Gln |
| Asp | Glu |
| Cys | Ser, Ala |
| Gln | Asn |
| Glu | Asp |
| Gly | Pro, Ala |
| His | Asn, Gln, Lys, Arg |
| Ile | Leu, Val, Met, Ala, Phe |
| Leu | Ile, Val, Met, Ala, Phe |
| Lys | Arg, Gln, Asn |
| Met | Leu, Phe, Ile |
| Phe | Leu, Val, Ile, Ala, Tyr |
| Pro | Ala |
| Ser | Thr, Ala, Cys |
| Thr | Ser |
| Trp | Tyr, Phe |
| Tyr | Trp, Phe. Thr, Ser |
| Val | Ile, Met, Leu, Phr, Ala |

Further variants include unusual or un-natural amino acids, peptide branches or other modifications. Any modification should preferably avoid low synthesis yields, and should avoid aggregation or poor solubility. Modified amino acids may by incorporated to enhance affinity or stability of secondary structures. Modified amino acids can routinely be incorporated into peptides synthesised by SPPS. Examples include D-amino acids, homo amino acids, beta-homo amino acids, N-methyl amino acids, alpha-methyl amino acids, non-natural side chain variant amino acids and other unusual amino acids (e.g. (Cit), hydroxyproline (Hyp), norleucine (Nle), 3-nitrotyrosine, nitroarginine, ornithine (Orn), naphtylalanine (Nal), Abu, DAB, methionine sulfoxide or methionine sulfone). For example, D-amino acids may be incorporated to increase resistance against degradation enzymes, homo-amino acids have an additional CH₂ attached to the alpha-carbon of the amino acid and may have improved biological activity or stability.

In some instances, the CPP moiety will be positioned closer to the N-terminus of the peptide conjugate than the therapeutic moiety. In other instances, the CPP moiety will be positioned closer to the C-terminus than the therapeutic moiety. Preferably, the CPP moiety will be positioned closer to the N-terminus of the peptide conjugate than the therapeutic moiety.

The ability of a naturally occurring or synthetic sequence to translocate the membrane may be tested by routine methods known in the art and illustrated in the accompanying examples.

In particular embodiments, the cell-penetrating peptide of the invention comprises at least one of the modifications listed below:
1. mutation of any arginine to lysine to increase the positive charge and improve the membrane translocation or stability properties;
2. mutation of any lysine to arginine or another tolerated residue to reduce the positive charge and improve the membrane translocation or stability properties;
3. replacement of Cys-39 with any tolerated residue to maintain or improve the membrane translocation or stability properties whilst reducing the presence of thiol groups;
4. introduction of an alternative thiol residues through mutagenesis at positions 1 to 60 to introduce an alternative thiol for chemical conjugation;
5. mutation of any non-basic residue into a basic residue to increase the pH of the protein to maintain or improve the membrane translocation or stability properties;
6. the introduction of any stabilising mutations, such as disulphide bridges, ionic interactions, hydrophobic interactions, reduced proteolysis liabilities in residues 1-60 to improve the membrane translocation or stability properties;
7. chemical modification of any residue 1-60, such as alkylation, cross-linking, stapling, etc to improve the membrane translocation or stability properties;
8. the addition of any chemical modification at the N-terminus or C-terminus to improve the chemical conjugation, membrane translocation or stability properties; and/or
9. the replacement of any natural amino acid (L-amino acid) with non-natural amino acids (D-amino acid) to improve the membrane translocation or stability properties.

In a preferred embodiment, the ANTP sequence or a fragment thereof may be modified to replace the residue corresponding to Cys-39 in SEQ ID NO:2 with a Ser or Ala. For example, the residue corresponding to Cys-39 in SEQ ID NO:2 may be replaced with a serine. Exemplary cell-penetrating peptides comprise or consist of the sequence of SEQ ID NO: 16 or a sequence with at least 80%, 85%, 90% or 95% sequence identity to SEQ ID NO:2 wherein the sequence also comprises the aforementioned C39S mutation.

Alternatively or additionally, the sequence of the cell penetrating peptide may be modified to add an N and/or C terminal cysteine. For example, SEQ ID NO: 2, SEQ ID NO:4 or SEQ ID NO: 16 may be modified by the addition a cysteine at the N and/or C terminus of said sequence. A cysteine may also be inserted into the N and/or C proximal region of SEQ ID NO: 2, SEQ ID NO:4 or SEQ ID NO: 16. Alternatively, where the cell penetrating peptide comprises or consists of a fragment of SEQ ID NO:2 or SEQ ID NO: 16, the fragment may be modified by the addition of an N and/or C terminal cysteine. In preferred embodiments, the cell penetrating peptide comprises or consists of any one of SEQ ID NO:13, SEQ ID NO:14, SEQ ID NO:15, or SEQ ID NO:16.

In a preferred embodiment, the aforementioned cell penetrating peptide is conjugated to a therapeutic protein to form a fusion protein. In a particularly preferred embodiment, the therapeutic protein is a dominant negative (DN) protein/cargo moiety as described below. For example, the cell penetrating peptide may be conjugated to a cargo protein via a thioester bond formed with the N or C terminal cysteine thiol of the ANTP.

### Dominant-Negative (DN) Peptides/Proteins

The “second component” in the therapeutic agent of the present invention is a dominant negative (DN) protein/cargo moiety. The cargo moiety may be any therapeutic peptide that is not naturally associated with the CPP moiety. In preferred embodiments, the cargo is an inhibitor of Notch signalling. The cargo moiety may be derived from a naturally occurring peptide. Alternatively, the cargo moiety may be engineered.

Dominant-negative (DN) mutations are gain-of-function mutations that lead to the production of mutated proteins that contribute to the formation of dimers or multimers that act in a dominant-negative fashion (dominant-negative effect (DNE)) to inhibit overexpressed or abnormally active pathways and/or to antagonise the action of abnormally expressed proteins (Herskowitz, 1987). Although Herskowitz' definition referred basically to intralocus interactions, it is now recognised that interlocus (e.g., trans-acting) interactions can also lead to dominance (Omholt et al., 2000).

DN mutations include mutations in the genes, whose products form multimeric complexes, either with themselves or with other proteins as well as DN mutations that appear in homodimeric ligands. Transcriptional regulation can also be the subject of a DNE. To achieve this type of DNE, transactivation domain of the modular transcription factor (TF) is removed, leaving only DNA binding domain behind. The resulting truncated TF will behave as a competitive inhibitor of the transcription.

In a preferred embodiment, the dominant-negative protein is an inhibitor of the Notch pathway. The term “Notch inhibitor” is intended to include any molecule that is able to reduce Notch signalling. Notch inhibitors can target any step in the Notch signalling pathway; including ligand-receptor binding, ADAM mediated cleavage, γ secretase mediated cleavage, Notch transcription complex assembly, or the expression of putative Notch target genes and proteins. Whether a molecule acts as a Notch inhibitor can be determined using standard molecular biology techniques. For example, the expression of putative Notch target genes (including Hes and Hey) in treated and control cells can be quantified by real time quantitative PCR (RT-qPCR), expression of a large number of Notch responsive genes can be quantified simultaneously using a Microarray, cleaved NICD can be visualised using labelled antibodies or in situ hybridisation, or transcriptional reporter assays utilising Notch-responsive promoters (based either on endogenous targets or on multimerised CSL-binding sites) can be used to control expression of fluorescent, bioluminescent, or other reporter proteins. NICD or NAECD gain of function cells have constitutively high NOTCH activity and are therefore useful in these studies.

The ability of a peptide to inhibit Notch signalling can be easily tested by a person skilled in this field. For example, the ability of a peptide to inhibit Notch signalling can be measured in vitro. A suitable method is described in the Examples in relation to MDA-MB-231 cells.

In preferred embodiments, the cargo moiety is derived from the co-activator Mastermind-like (MAML) protein. MAML is highly conserved. Therefore, any MAML homolog may be used in the present invention. The MAML derivative used in the invention should be able to bind to at least one of NICD or CBF-1. The MAML derivative used in the invention should also inhibit assembly of a functional Notch transcriptional complex.

Described herein are MAML (dnMAML) variants that may be used in the peptide conjugate of the invention. For example, one preferred embodiment utilises a 62-amino-acid MAML truncation known as dnMAML(13-74) (SEQ ID NO:9). The kinked alpha-helix of MAML(13-74) forms a stable ternary complex with CBF-1 and NICD. Since SEQ ID NO:9 lacks the C-terminal portion necessary for functional Notch transcriptional complex assembly, MAML(13-74) is a dominant-negative truncation. This peptide has been shown to be effective in inhibiting Notch signalling and the growth of tumors. Thus, in some embodiments the peptide conjugate comprises a cargo moiety comprising SEQ ID NO:9 or suitable variants thereof.

In one embodiment, the “second component” comprises or consists of a DN human Mastermind-like protein (MAML). The 62 amino acid sequence of DN-MAML (SEQ ID NO: 9) is shown below.

L P R H S A V M E R L R R R I E L C R R H H S T C E A R Y E A
V S P E R L E L E R Q H T F A L H Q R C I Q A K A K R A G K H

Alternatively, DN-MAML may be modified by mutagenesis or chemical modification to improve its inhibitory, stability or manufacturing properties. The chemical modifications can be as described above with regards to the CPP. In one example, the peptide is a stapled peptide. For example, the peptide may be a stapled peptide derived from an alpha-helix in Mastermind-like (MAML) protein.

The solved crystal structure of the CBF-1-NICD-MAML ternary complex identified the residues that participate in transcriptional complex formation. These residues are underlined in the below sequence (SEQ ID NO:9) and should be retained in dnMAML variants of the invention:

LPRHSAVMERLRRRIELCRRHHSTCEARYEAVSPERLELERQHTFALHQ
RCIQAKAKRAGKH

The remaining amino acid residues may be replaced. Preferably, amino acid substitutions will be conservative in nature. The skilled person will be able to determine whether a given amino acid substitution will be conservative using common general knowledge and the information in Table 1. In some embodiments, the cargo moiety comprises an amino acid sequence that is at least 60%, at least 70%, at least 80%, at least 85%, at least 90%, at least 95%, at least 98% identical to SEQ ID NO:9. For example, in some instances, the cargo moiety comprises an amino acid sequence that is at least 80% identical to SEQ ID NO:9. In other instances, the cargo moiety comprises an amino acid sequence that is at least 90% identical to SEQ ID NO:9. In other instances, the cargo moiety comprises an amino acid sequence that is at least 95% identical to SEQ ID NO:9. In other instances, the cargo moiety comprises an amino acid sequence that is at least 98% identical to SEQ ID NO:9. In preferred instances, the cargo moiety comprises an amino acid sequence that is SEQ ID NO:9.

In preferred embodiments, the cargo moiety is derived from human MAML. In other instances, the cargo moiety may be derived from any MAML homolog. A variant cargo moiety may comprise an equivalent sequence derived from a different organism. For example, a dnMAML variant may comprise any peptide that is equivalent to amino acids 13 to 74 of the human MAML sequence but derived from the MAML gene of a different organism. Such a species variant may derive from any organism that expresses a MAML protein. For example, the species variant may derive from a mammal such as a primate, rodent or a domestic or farm animal. A variant peptide may also comprise a variant of such a species variant sequence such as a deletion, addition or substitution variant as described herein.

Further variants include modified, unusual or unnatural amino acids. Amino acids suitable for use in the present invention are described above and include D-amino acids, homo amino acids, beta-homo amino acids, N-methyl amino acids, alpha-methyl amino acids, non-natural side chain variant amino acids and other unusual amino acids (e.g. (Cit), hydroxyproline (Hyp), norleucine (Nle), 3-nitrotyrosine, nitroarginine, ornithine (Orn), naphtylalanine (Nal), Abu, DAB, methionine sulfoxide or methionine sulfone).

The production and generation of a peptide/protein that acts in a dominant-negative way can be achieved by the routine DNA recombinant techniques. To demonstrate that a protein truly acts in a dominant-negative fashion, a number of standard techniques can be used. The most commonly used way of validating if a protein acts in a dominant-negative fashion is Western blot analysis of cell lysates that were treated with the invention and negative controls.

### Linkers

The “third component” of the invention is the linker connecting ANTP and the DN protein cargo. This linker can be a peptide fusion in any orientation or the result of chemical conjugation approaches to link the two components at any position 1-60 of ANTP to any position 1-62 of DN-MAML. The conjugation or fusion ratio can comprise more than one of either components.

Individual proteins or peptides can be connected directly as a recombinant fusion protein, with or without an interconnecting peptide linker. Proteins or peptides can be connected using chemical modifications to each component resulting in a chemical ligation. Linkers or spacers are short amino acid sequences created to separate multiple domains in a protein. Most of them are rigid and have the function to prohibit unwanted interactions between the discrete domains. Gly-rich linkers, on the other hand, are flexible, connecting various domains in a single protein without interfering with the function of each domain. The advent of recombinant DNA technology made it possible to fuse two interacting partners with the introduction of artificial linkers. Often, independent proteins may not exist as stable or structured proteins until they interact with their binding partner, following which they gain stability and the essential structural elements. Gly-rich linkers have been proven useful for these types of unstable interactions, particularly where the interaction is weak and transient, by creating a covalent link between the proteins to form a stable protein-protein complex. Gly-rich linkers are also employed to form stable covalently linked dimers and to connect two independent domains that create a ligand-binding site or recognition sequence. The lengths of linkers vary from 2 to 31 amino acids, optimized for each condition so that the linker does not impose any constraints on the conformation or interactions of the linked partners.

Linkers described in the art are generally classified into 3 categories according to their structures: flexible linkers, rigid linkers, and in vivo cleavable linkers. Besides the basic role in linking the functional domains together (as flexible and rigid linkers) or releasing the free functional domain in vivo (as in vivo cleavable linkers), linkers may offer many other advantages for the production of fusion proteins, such as improving biological activity, increasing expression yield, and achieving desirable pharmacokinetic profiles. The structure and design of useful linkers have been reviewed by Chen et al (2013).

The term “flexible linker” used herein refers to linkers that are composed of small polar (e.g. glycine) and non-polar (e.g. serine and threonine) amino acids that allow a certain degree of flexibility and mobility between the two functioning domains. It is the small size of the amino acids that allows the flexibility and mobility of the connecting functional domains. The presence of serine and threonine stretches in linker proteins ensures the stability of the linker proteins in aqueous solutions, as these amino acids form hydrogen bonds with water molecules that reduce the unfavourable interaction between the linker and the protein moieties.

The most commonly used flexible linkers consist of mainly glycine and serine residues (e.g. (Gly-Gly-Gly-Gly-Ser)n, (SEQ ID NO:18) where n can be adjusted to maintain necessary interdomain interactions). Linkers that are composed of only glycine residues, such as (Gly)₈ can be used to increase the accessibility of epitopes to antibody and/or to improve protein folding. Gly and Ser rich flexible linker, GSAGSAAGSGEF (SEQ ID NO: 19), that was designed by Waldo and colleagues is a useful linker with good solubility. Moreover, the DNA sequence of this linker does not have high homologous repeats, which protects it from being deleted during homologous recombination during shuffling protocol for cloning.

In some instances, the use of flexible linkers might not be favourable. Flexible linkers might cause the failure of the experiment due to inefficient separation of the functional domains and subsequent insufficient reduction of their interference with each other. Rigid linkers can be used to overcome these limitations that are associated with flexible linkers. Rigid linkers, on the other hand, provide a fixed distance between the functioning domains and allow the maintenance of their independent functions. The empirical formula of rigid linkers was first disclosed by Arai (2004). Conformations of variably linked chimeric proteins were evaluated by synchrotron X-ray small-angle scattering. Proline-rich linkers are also a type of rigid linkers.

The third type of linkers is in vivo cleavable linkers. Use of in vivo cleavable linkers has a number of benefits including prevention of steric hindrance between the functional domains and lack of altered biodistribution and metabolism of the protein moieties due to the interference between domains. Another advantage of in vivo cleavable linkers is that they allow the delivery of prodrugs to target sites where the linkers are processed to activate bioactivity.

The reversible nature of disulphate bonds was utilised in the design of an in vivo cleavable linker by Chen and colleagues. This linker allows generating a precisely constructed, homogeneous product by recombinant methods. Additionally, there are linkers that are cleavable by small molecules include Met-X sites, cleavable by cyanogen bromide, Asn-Pro, cleavable by a weak acid, Trp-X cleavable by among other things, NBS-skatole and the others. Due to the milder conditions that are required for the cleavage, protease-cleavable linkers are preferred. The selection of the cleavage site should not be a problem for a skilled scientist as all of the sequences, such as the cleavage site that is targeted by factor Xa, Enterokinase or Thrombin are accessible.

There are online tools and databases available for linker protein design, which allows the design of the best-suited linker protein that meets the unique requirements of for the fusion protein generation. LINKER program is an example of digital tools that searches its database for the most suitable linkers for an experiment based on the user-specified input. The second database is run by the Centre for Integrating Bioinformatics VU (IBIVU) at Vrije University of Amsterdam.

An alternative way of connecting two functional proteins is by chemical conjugation. Each partner protein is chemically modified to contain reactive moieties which form a covalent bond upon contact. There are many strategies for protein bioconjugation, which have been described in ‘Bioconjugation: Methods and protocols, (Mass & Devoogdt Eds), Methods in Molecular Biology (2019) and Bioconjugate Techniques (Hermanson, Ed) Academic Press, 2008.

Examples of protein chemical bioconjugation approaches include:
1. Activated-esters coupling of lysine residues;
2. Methanesulfonyl acrylate coupling of lysine residues;
3. Copper and non-catalysed ‘click’ reactions of alkynes introduced into proteins;
4. Disulphide bonds formed with native or engineered cysteine thiol groups;
5. Thioether bonds formed with cysteine thiols and introduced maleimide groups;
6. Native chemical ligation;
7. Smartag chemo-selective ligation using formyl-glycine modified proteins;
8. Chemoselective Azo-coupling reactions;
9. Cyclo-addition reactions (Cu-AAC or SPAAC) of azide-derivatized amino acids;
10. Photo-reactive or photo-catalysed chemical reactions.

Further examples of the chemically reactive groups available for bioconjugation include:
1. Amine Reactions (Isothiocyanates, Isocyanates, Acyl Azides, NHS Esters, Sulfonyl Chlorides, Aldehydes, Glyoxals, Epoxides, Oxiranes, Carbonates, Arylating Agents, Imidoesters, Carbodiimides, Anhydrides, Fluorophenyl Esters, Hydroxymethyl Phosphine Derivatives, Guanidination of Amines);
2. Thiol Reactions (Haloacetyl and Alkyl Halide Derivatives, Maleimides, Aziridines, Acryloyl Derivatives, Arylating Agents, Thiol-Disulfide Exchange Reagents (Pyridyl Disulfides, TNB-Thiol, Disulfide Reductants), Vinylsulfone Derivatives, Metal-Thiol Dative Bonds, Native Chemical Ligation, Cisplatin Modification of Methionine and Cysteine;
3. Carboxylate Reactions (Diazoalkanes and Diazoacetyl Compounds, Carbonyl-diimidazole, Carbodiimides);
4. Hydroxyl Reactions (Epoxides and Oxiranes, Carbonyldiimidazole, N,N-Disuccinimidyl Carbonate or N-Hydroxysuccinimidyl Chloroformate, Oxidation with Periodate, Enzymatic Oxidation, Alkyl Halogens, Isocyanates); [symbol]
5. Aldehyde and Ketone Reactions (Hydrazine Derivatives, Schiff Base Formation, Reductive Amination, Mannich Condensation);
6. Active Hydrogen Reactions (Diazonium Derivatives, Mannich Condensation, Iodination Reactions);
7. Photo-Chemical Reactions (Aryl Azides and Halogenated Aryl Azides, Benzophenones, Anthraquinones, Certain Diazo Compounds Diazirine Derivatives, Psoralen Compounds);
8. Cycloaddition Reactions (Diels-Alder Reaction, Complex Formation with Boronic Acid Derivatives, Click Chemistry: Cu¹-promoted Azide-Alkyne [3 2] Cycloaddition).

Examples of the types of protein chemical cross-linkers than can be employed include:
1. Zero-Length Crosslinkers: Carbodiimides (EDC, EDC plus Sulfo-NHS, CMC, DCC, DIC), Woodward's Reagent K, N,N-Carbonyldiimidazole, Schiff Base Formation and Reductive Amination)
2. Homobifunctional Crosslinkers: Homobifunctional NHS Esters (DSP and DTSSP, DSS and BS3, DST and Sulfo-DST, BSOCOES and Sulfo-BSOCOES, EGS and Sulfo-EGS, DSG, DSC), Homobifunctional Imidoesters (DMA, DMP, DMS, DTBP), Homobifunctional Sulfhydryl-Reactive Crosslinkers (DPDPB, BMH), Difluorobenzene Derivatives (DFDNB, DFDNPS), Homobifunctional Photoreactive Crosslinkers (BASED), Homobifunctional Aldehydes (Formaldehyde, Glutaraldehyde), Bis-epoxides (1,4-Butanediol Diglycidyl Ether), Homobifunctional Hydrazides, Adipic Acid, Dihydrazide, Carbohydrazide), Bis-diazonium Derivatives (o-Tolidine, Diazotized, Bis-diazotized Benzidine), Bis-alkyl Halides [symbol]
3. Hetero-bifunctional Crosslinkers: Amine-Reactive and Sulfhydryl-Reactive Crosslinkers (SPDP, LC-SPDP, and Sulfo-LC-SPDP, SMPT and Sulfo-LC-SMPT, SMCC and Sulfo-SMCC, MBS and Sulfo-MBS, SIAB and Sulfo-SIAB, SMPB and Sulfo-SMPB, GMBS and Sulfo-GMBS, SIAX and SIAXX, SIAC and SIACX, NPIA), Carbonyl-Reactive and Sulfhydryl-Reactive Crosslinkers (MPBH, M2C2H, PDPH), Amine-Reactive and Photoreactive Crosslinkers (NHS-ASA, Sulfo-NHS-ASA, and Sulfo-NHS-LC-ASA, SASD, HSAB and Sulfo-HSAB, SANPAH and Sulfo-SANPAH, ANB-NOS, SAND, SADP and Sulfo-SADP, Sulfo-SAPB, SAED, Sulfo-SAMCA, p-Nitrophenyl Diazopyruvate, PNP-DTP), Sulfhydryl-Reactive and Photoreactive Crosslinkers (ASIB, APDP, Benzophenone-4-iodoacetamide, Benzophenone-4-maleimide), Carbonyl-Reactive and Photoreactive Crosslinkers (ABH), Carboxylate-Reactive and Photoreactive Crosslinkers (ASBA), Arginine-Reactive and Photoreactive Crosslinkers (APG)
4. Trifunctional Crosslinkers (4-Azido-2-nitrophenylbiocytin-4-nitrophenyl ester, Sulfo-SBED, MTS-ATF-Biotin and MTS-ATF-LC-Biotin, Hydroxymethyl Phosphine Derivatives)

Enzyme-catalysed conjugation methods include sortase-mediated, transglutamase-mediated, trypsiligase-mediated, myristoyltransferase-mediated, tyrosine-ligase-mediated, lipoicnacid ligase-mediated, farnesyltransferase-mediated, enzymic modification of protein glycans (chemo-enzymic glycoengineering).

Fusing CPPs with dominant-negative protein/peptide as described herein allows the design of a therapeutic molecule that can penetrate all cell types and effectively inhibiting/preventing the function of intracellular targets. These features enable the invention to be a more effective therapeutic agent.

In a preferred embodiment, the cell penetrating peptide is conjugated to a DN-MAML protein via a thioester bond formed with a thiol in an additional cysteine at the N and/or C terminus of ANTP. The DN-MAML protein may be modified to include a maleimide group.

### Medical Use

In one embodiment, the present invention provides an agent comprising a CPP chemically or recombinantly conjugated to a dominant-negative protein/peptide, for use in therapy. In one embodiment, the CPP is a derivative of ANTP. In one embodiment, the dominant-negative protein/peptide is a protein/peptide comprising a mutation that act in a dominant-negative fashion (e.g. DN-MAML, AKT-DN, STAT3). In one embodiment, the dominant negative proteins/peptides of the invention inhibit the Notch pathway which is overactive in numerous types of cancers.

The agent of the invention may be used in a method of treating cancer. In a preferred embodiment, the agent of the invention may be used in a method of treating cancer, wherein the cancer is characterised by the aberrant activation of the Notch signalling pathway. Aberrant activation of the Notch signalling leads to the pathogenesis of different human malignancies, including acute T-cell lymphoblastic leukaemia, lymphomas, advanced renal cell carcinoma, cervical, prostate tumours and glioblastomas, lung, pancreas and breast cancers. Administration of the therapeutic agent of the invention may block the Notch pathway, thereby slowing the growth of tumours, reduced cell proliferation and downregulation of NOTCH target genes (e.g., HES7 and HEY1).

Also provide is a method of treating cancer, the method comprising administering an effective amount of the therapeutic agent to a subject in need thereof.

Also provided is the use of a therapeutic agent, as defined above, in the manufacture of a medicament for the treatment of cancer.

### Pharmaceutical Compositions

The cell-penetrating peptide or fusion protein of the invention may be formulated as a pharmaceutical composition. The pharmaceutical composition may be used in a method of therapy, and in particular, in a method or treating or preventing a disease, disorder or symptom linked to aberrant Notch signalling. For example, the pharmaceutical composition may be used in a method of treating cancer. The pharmaceutical composition of the invention may additionally or alternatively be used in the manufacture of a medicament for treating cancer. The invention further provides a method of treating or preventing cancer, wherein the method comprises administering to a subject in need thereof a pharmaceutical composition comprising a peptide conjugate of the invention.

Formulation of a composition comprising the cell-penetrating peptide or fusion protein of the invention can be carried out using standard pharmaceutical formulation chemistries and methodologies all of which are readily available to the reasonably skilled artisan. The composition of the invention comprises, in addition to the peptide conjugate of the invention, a pharmaceutically acceptable carrier. As used herein, the term “pharmaceutical carrier” covers all types of solvents, dispersion media, coatings, antibacterial and antifungal agents, isotonic and absorption delaying agents and the like that are physiologically compatible. In particular, the composition may comprise at least one of: a pharmaceutically acceptable solvent, excipient or auxiliary compound. The solvents, excipients, and auxiliary substances are generally pharmaceutical agents that do not induce an immune response in the individual receiving the composition, and which may be administered without undue toxicity. The choice of pharmaceutically acceptable solvent, excipient or auxiliary compound will depend on the intended route of administration, standard pharmaceutical practice, and the known art. A thorough discussion of pharmaceutically acceptable excipients, vehicles and auxiliary substances is available in Remington's Pharmaceutical Sciences (Mack Pub. Co., N.J. 1991).

Pharmaceutically acceptable solvents useful for formulating an agent for administration to a subject are well known in the art. Preferred compositions for parenteral administration (i.e. intravenous bolus, intravenous infusion, intramuscular, intraperitoneal or subcutaneous injection) are in the form of a sterile aqueous solution such as water, physiologically buffered saline, or Ringer's solution. Other solvents that may be used include glycols, glycerol, oils such as olive oil or injectable organic esters. Compositions for parenteral administration may optionally contain other substances, for example, salts or monosaccharides to ensure the composition is isotonic with blood.

Alternatively, the cell-penetrating peptide or fusion protein of the invention may be encapsulated, adsorbed to, or associated with, particulate carriers. Suitable particulate carriers include those derived from polymethyl methacrylate polymers, as well as PLG microparticles derived from poly(lactides) and poly(lactide-co-glycolides). See, e.g., Jeffery et al. (1993) Pharm. Res. 10:362-368. Other particulate systems and polymers can also be used, for example, polymers such as polylysine, polyarginine, polyornithine, spermine, spermidine, as well as conjugates of these molecules.

Compositions for sustained release or implantation may comprise pharmaceutically acceptable polymeric or hydrophobic materials such as an emulsion, an ion exchange resin, a sparingly soluble polymer, or a sparingly soluble salt.

The composition of the drug can also contain adjuvants like preservatives, wetting agents, emulsifying agents. To prevent the presence of microorganisms, sterilisation procedures, and addition of various antibacterial and antifungal agents (parabens, chlorobutanol, phenols, sorbic acid and others alike) can be applied. It can also be useful to add such isotonic solutions like sugars, sodium chloride and the like into the composition. Additionally, aluminium monostearate and gelatine can be added to delay absorption.

The pharmaceutical compositions may be prepared, packaged, or sold in the form of a sterile injectable aqueous or oily suspension or solution. Injectable compositions may be prepared, packaged, or sold in unit dosage form, such as in ampoules or in multi-dose containers containing a preservative. In another embodiment, the active ingredient is provided in dry or lyophilised (e.g., a powder or granules) form for reconstitution with a suitable vehicle (e. g., physiologically buffered saline) prior to parenteral administration of the reconstituted composition.

Once formulated the compositions can be delivered to a subject in vivo using a variety of known routes and techniques. For example, a composition can be provided as an injectable solution, suspension or emulsion in oily or aqueous vehicles and administered via parenteral, subcutaneous, epidermal, intradermal, intramuscular, intra-arterial, intraperitoneal, intravenous injection using a conventional needle and syringe, or using a liquid jet injection system. Solutions, suspensions or emulsions may also be administered by a finely divided spray suitable for respiratory or pulmonary administration. If the peptide conjugate of the invention is formulated as a paste or implantable sustained-release or biodegradable formulation, the compositions may be administered topically to skin or mucosal tissue, such as nasally, intratracheally, intestinal, rectally or vaginally. Other modes of administration include oral administration, suppositories, and active or passive transdermal delivery techniques. A suitable route of administration may be determined by the skilled practitioner depending upon the particular symptom, disease or condition to be treated. Administration may be local to the site or tissue of interest, or may be systemic. An appropriate effective amount can be readily determined by one of skill in the art. Such an amount will fall in a relatively broad range that can be determined through routine trials. The compositions may contain from about 0.1% to about 99.9% of the peptide conjugate and can be administered directly to the subject or, alternatively, delivered ex vivo, to a sample derived from the subject, using methods known to those skilled in the art.

The preferred routes of administration are intravenous, intramuscular, subcutaneous, parental, spinal or epidermal administration. Therefore, the carrier should be suitable for these preferred routes of administration. “Parental administration” and “administered parentally” as used herein refers to modes of administration different from enteral and topical administration, usually by injection, and includes, without limitation, intravenous, intramuscular, intraarterial, intrathecal, intracapsular, intraorbital, intraperitoneal, transtracheal, subcutaneous, subcuticular, intraarticular, subcapsular, subarachnoid, intraspinal, epidural and intrastenal injection and infusion. In specific circumstances, the pharmaceutical compositions can also be administered by one or more of inhalation (suppository or pessary), topically (in a form of lotion, cream, ointment, dusting powder, skin patch), orally in the form of tablets containing starch or lactose as excipients, or in capsules alone or in admixture with excipients, or in the form of elixirs, solutions or suspensions containing flavourings or colouring agents.

The cell-penetrating peptide or fusion protein are administered to a subject in an amount that is compatible with the dosage formulation and that will be therapeutically effective. An appropriate effective amount will fall in a relatively broad range but can be readily determined by one of skill in the art by routine trials. The “Physicians Desk Reference” and “Goodman and Gilman's The Pharmacological Basis of Therapeutics” are useful for the purpose of determining the amount needed. As used herein, the term “therapeutically effective dose” of a peptide of the invention means a dose in an amount sufficient to reduce

The cell-penetrating peptide or fusion protein of the invention can be administered alone or together with other treatment or part of the treatment. In one embodiment, the pharmaceutical composition also can contain one or more additional auxiliary compound, such as a diagnostic reagent, nutritional substance, toxin, or therapeutic agent, for example, a cancer chemotherapeutic agent and/or vitamin(s).

Pharmacokinetic, pharmacodynamics and toxicology studies can be performed using numerous standard techniques. As described in the aforementioned studies of the Syntana-4 drug (Antennapedia-dominant negative mastermind-like construct) were performed in combination with innovative imaging including in vivo flow cytometry and whole-body fluorescence reflectance in an orthotopic model of breast cancer in SCID mice based on the implantation of MDA-MB-231 cells into mammary fat pads.

### EXAMPLES

The following examples are provided to further illustrate the advantages and features of the present invention, but are not intended to limit the scope of the invention. While they are typical of those that might be used, other procedures, methodologies, or techniques known to those skilled in the art may alternatively be used.

### Example 1: ANTP can Successfully Deliver Tumour-Suppressor Protein p21 into the Cancer Cells

ANTP can successfully deliver tumour-suppressor protein p21 into the cancer cells, where its expression is depleted. The resulting therapeutic protein (AB1, previously known as TR1) is disclosed in U.S. Pat. No. 10,259,852 B2. This novel cancer treatment was based on the published evidence that in many cancers, the expression of tumour suppressors such as p21 and p53 that regulate the cell cycle is impaired, which causes uncontrolled cellular division.

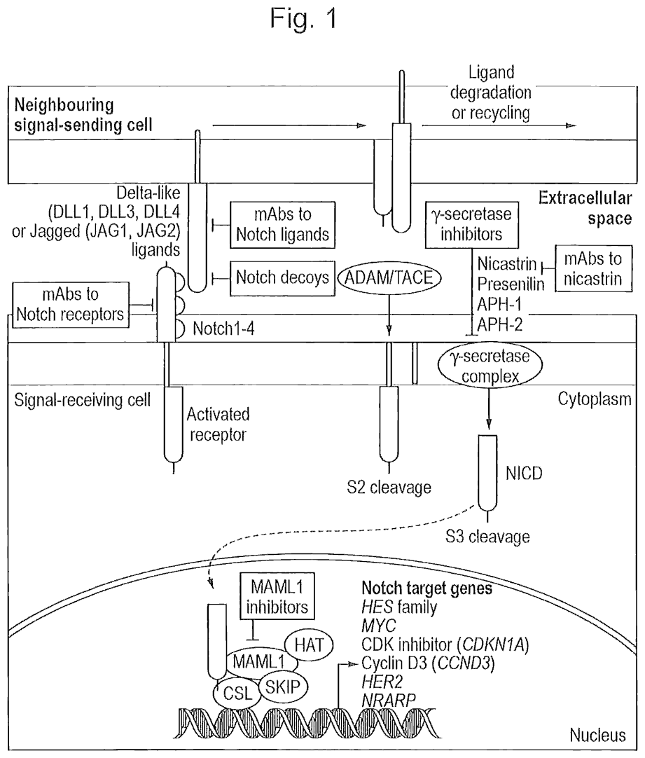

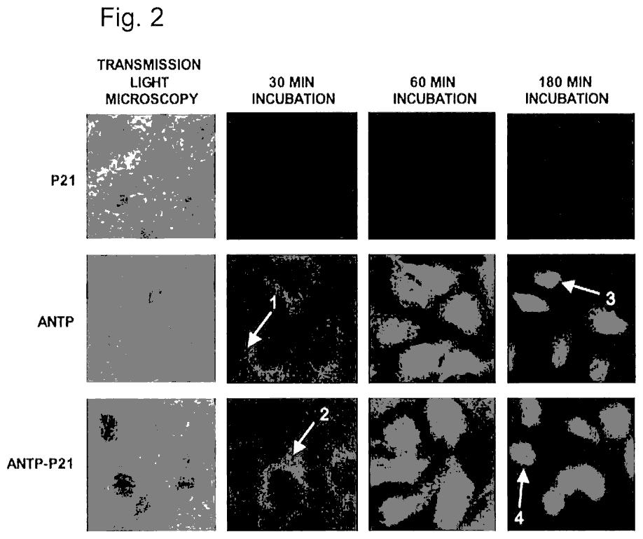

ANTP and ANTP-p21 were shown to penetrate cancer cells at a 100 μg/ml concentration (representative cells are shown in FIG. 1) trafficking to the nucleus within 1-3 hours. Cells, into which the protein had penetrated intranuclearly, were scored positive by immunofluorescence. No cellular uptake was seen with free p21 protein alone demonstrating that ANTP mediated the cellular delivery. Moreover, ANTP-p21 was demonstrated to kill the malignant cells selectively and did not elicit an immunogenic response in normal New Zealand White rabbits (Kousparou et al., 2012).

### Example 2: Recombinant Production of ANTP-MAML (TR4)

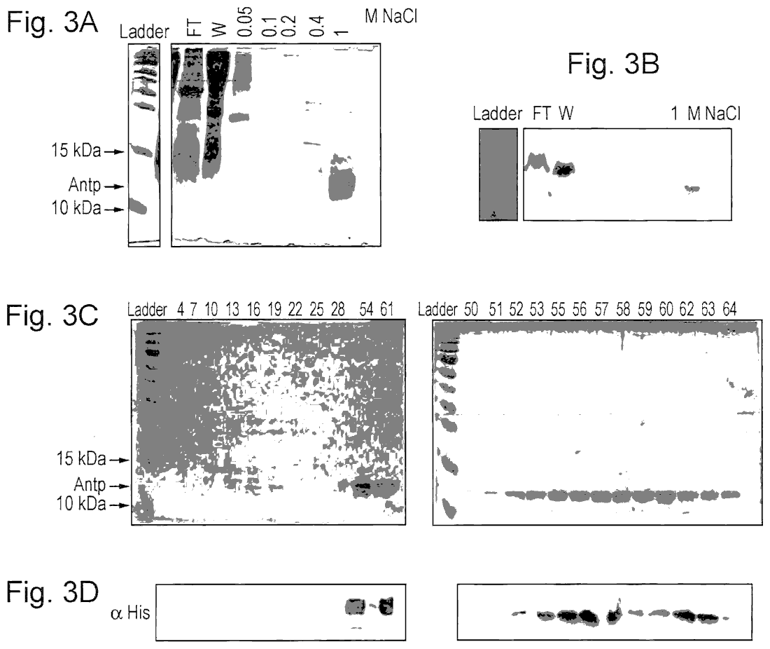

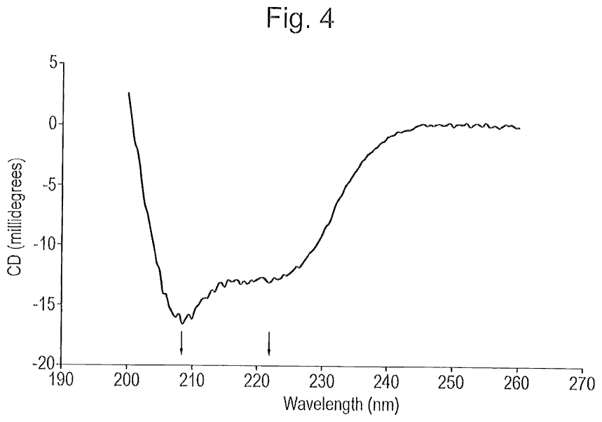

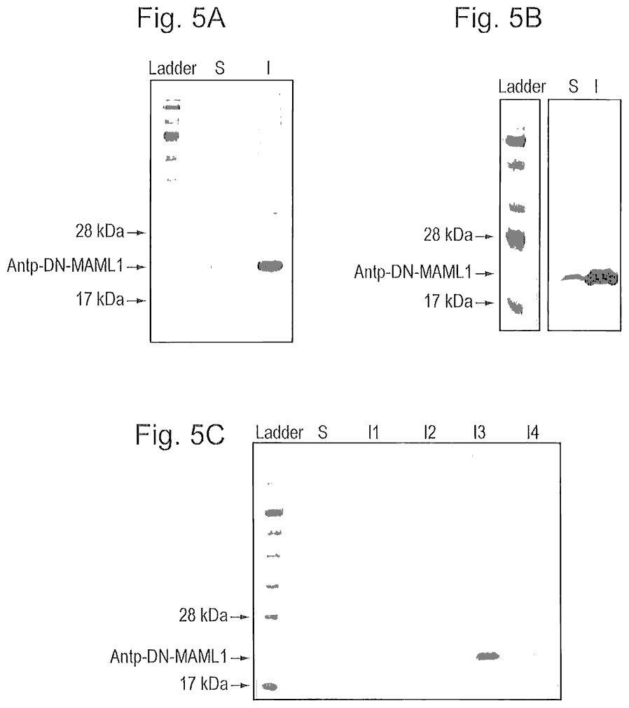

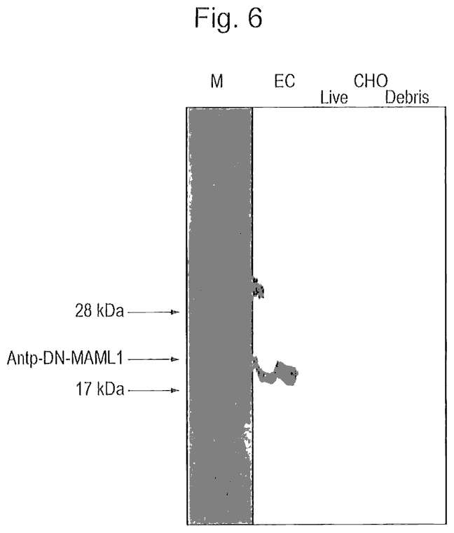

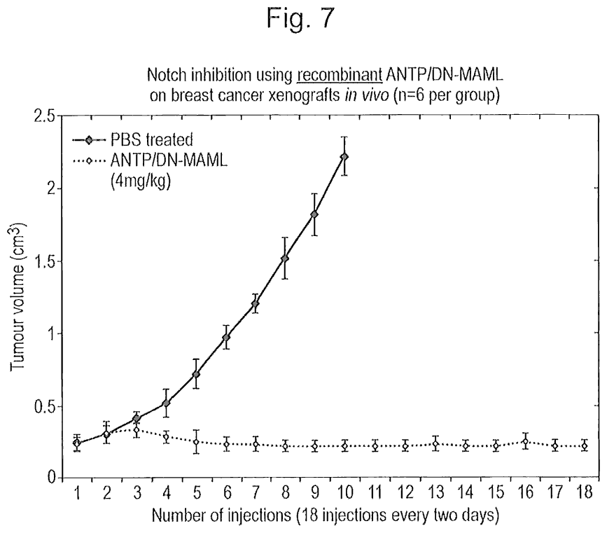

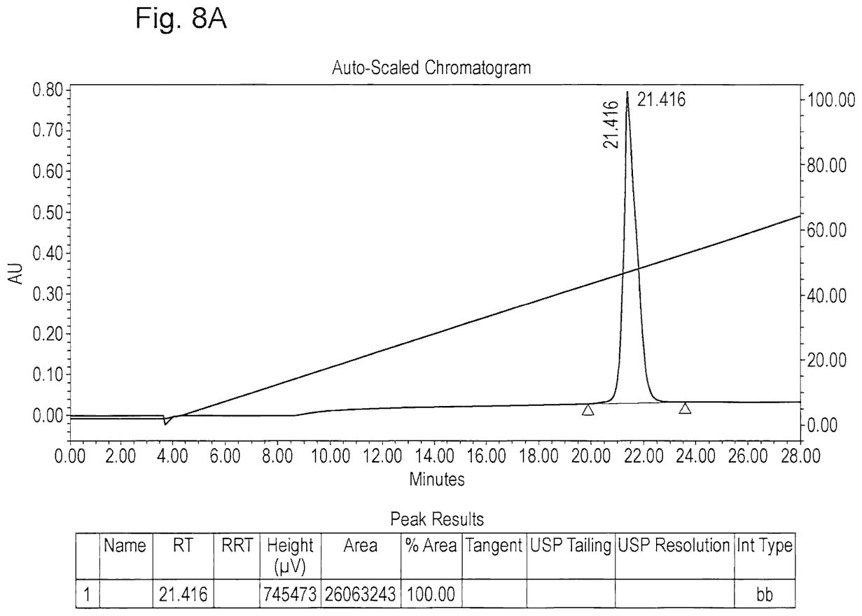

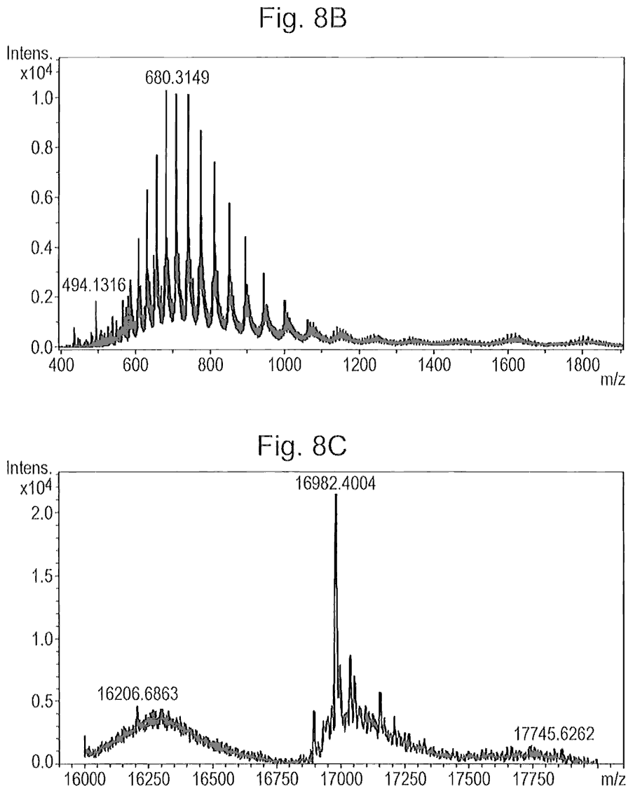

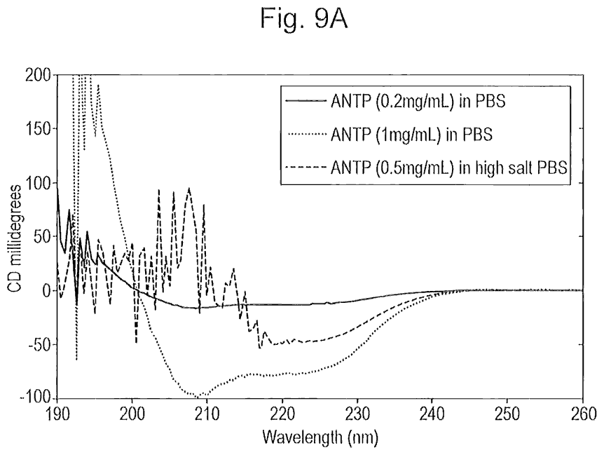

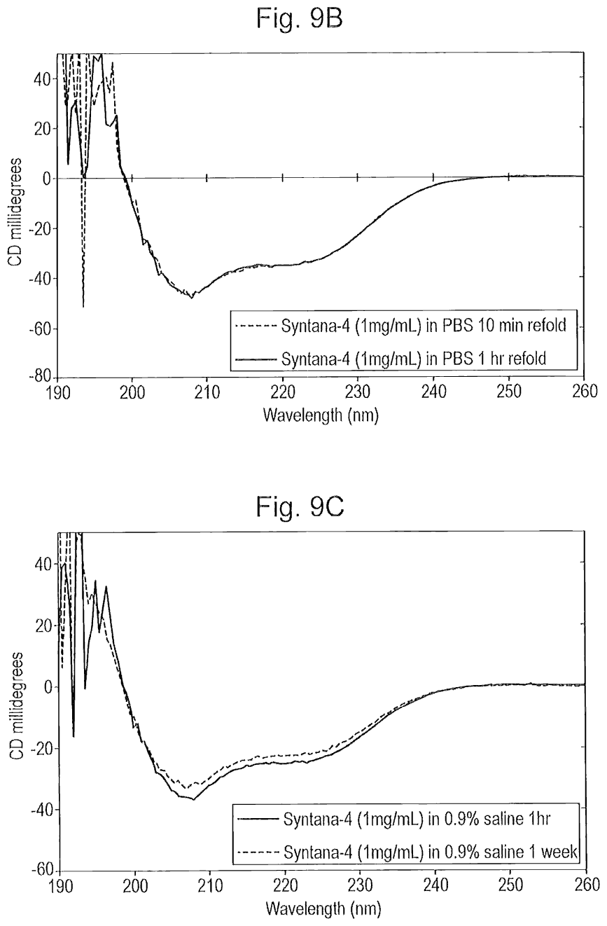

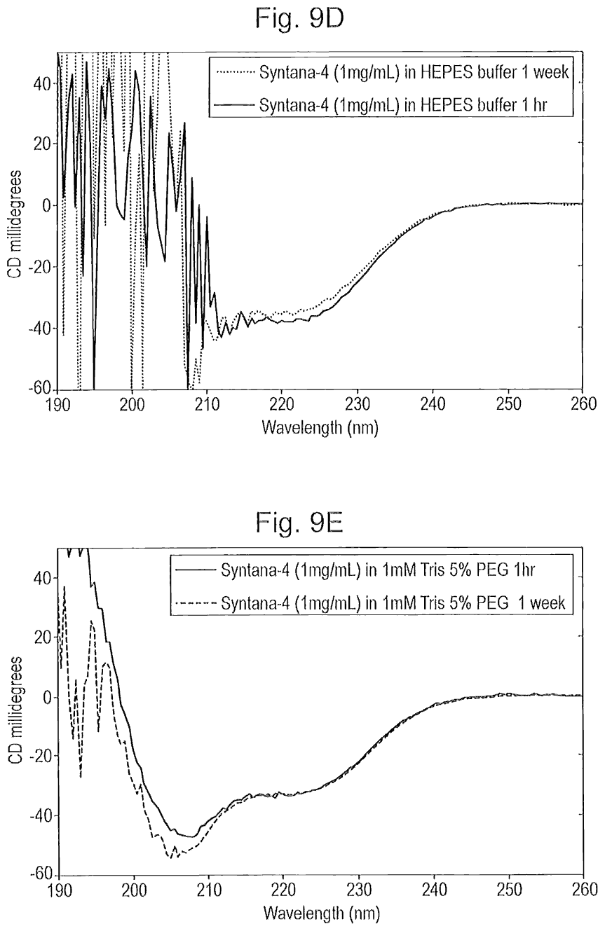

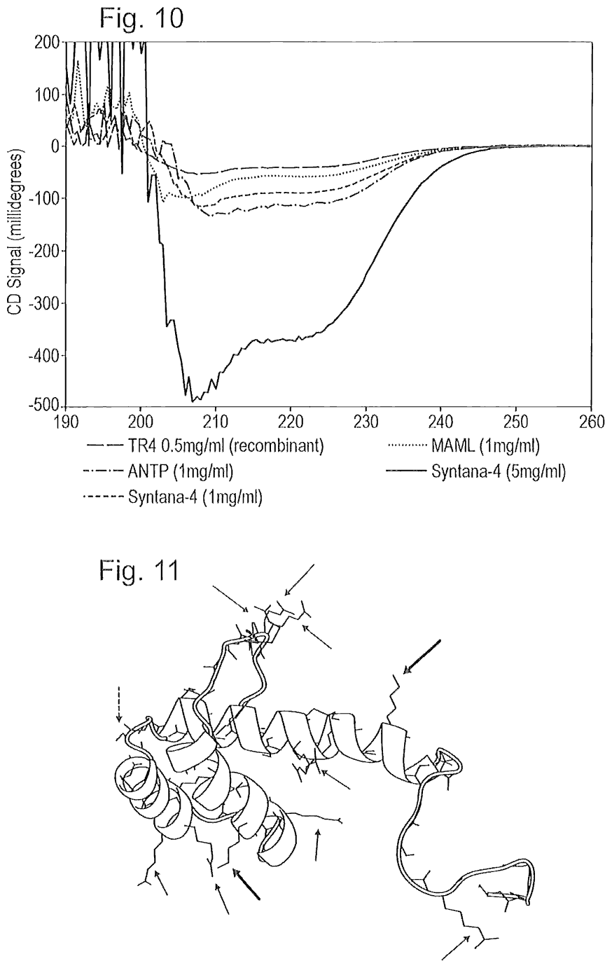

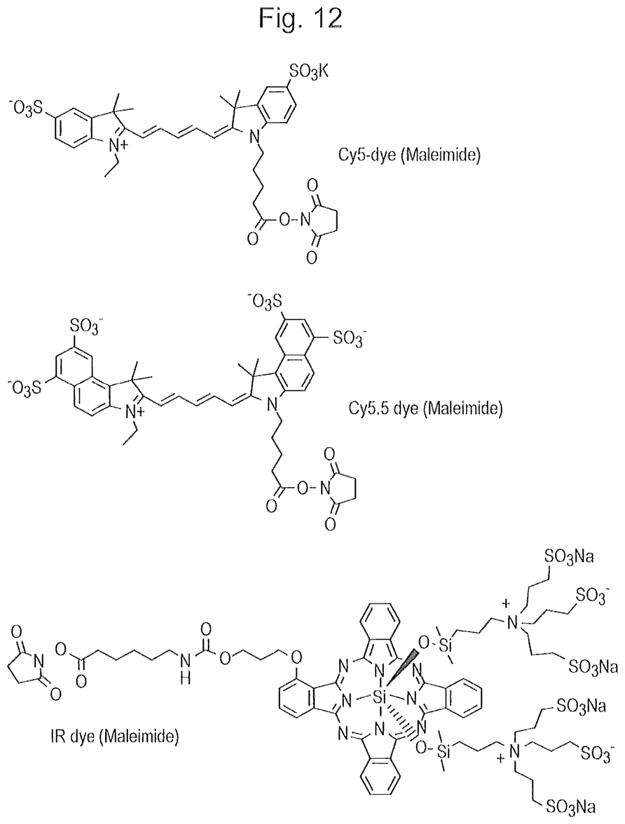

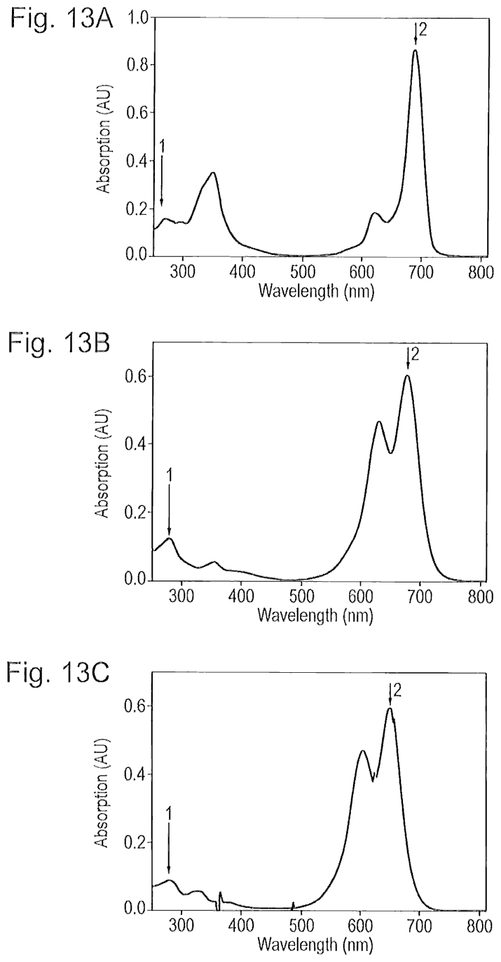

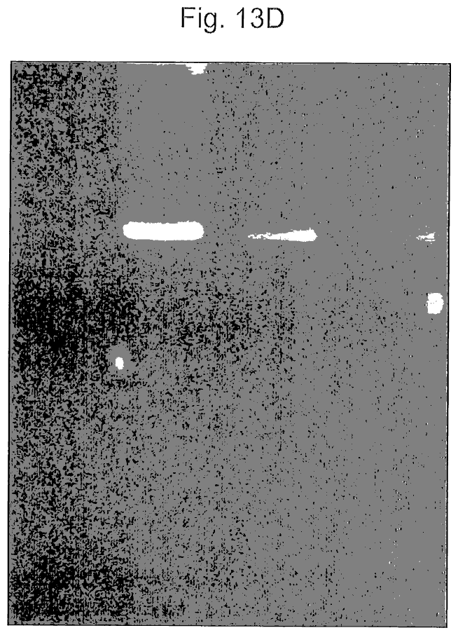

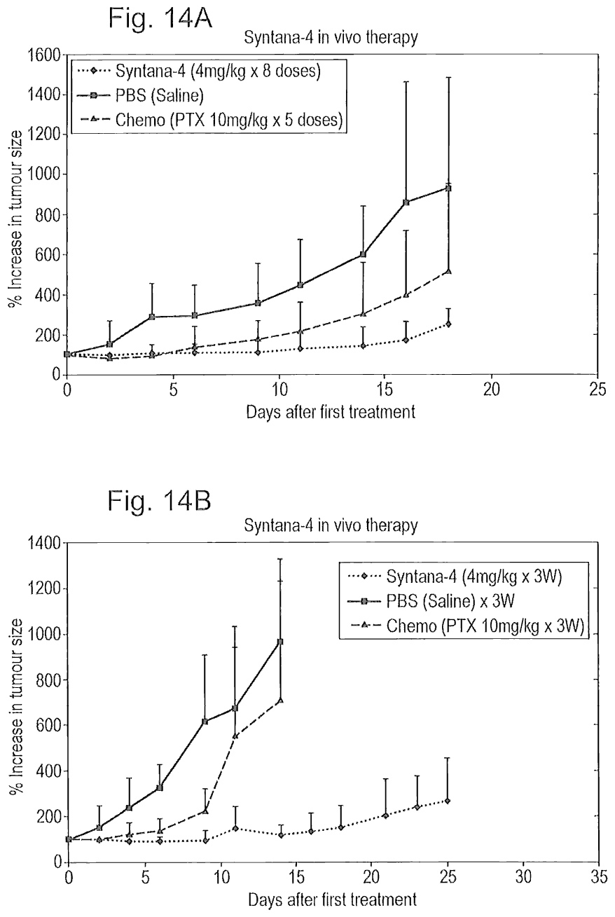

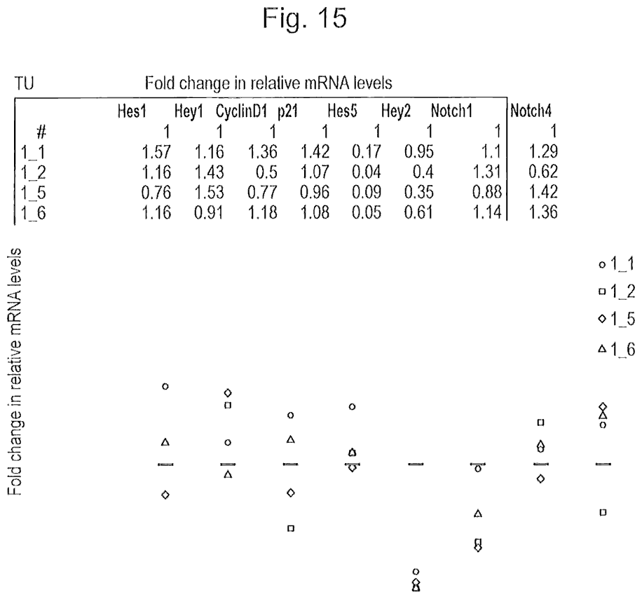

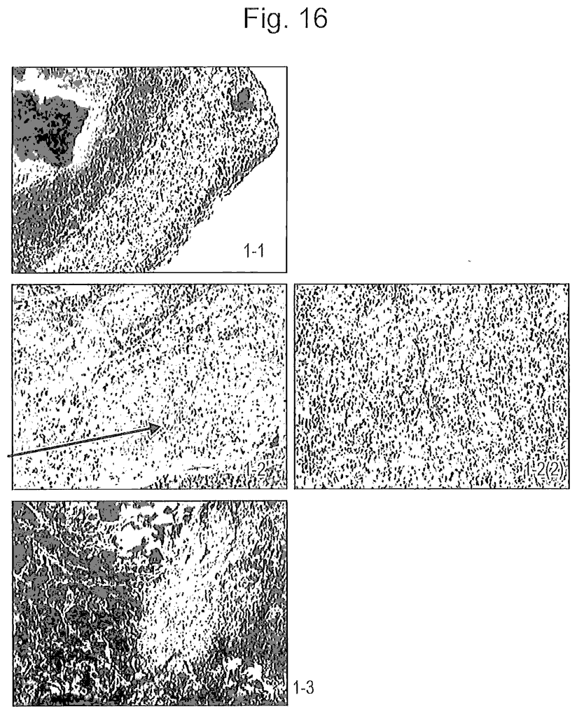

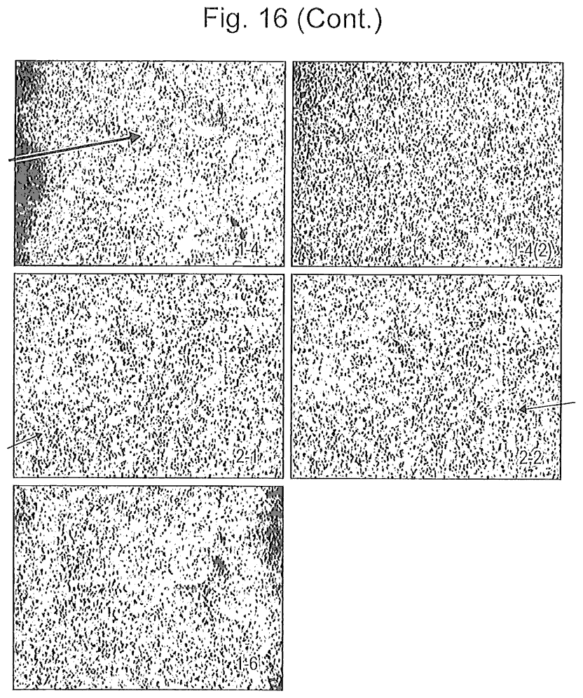

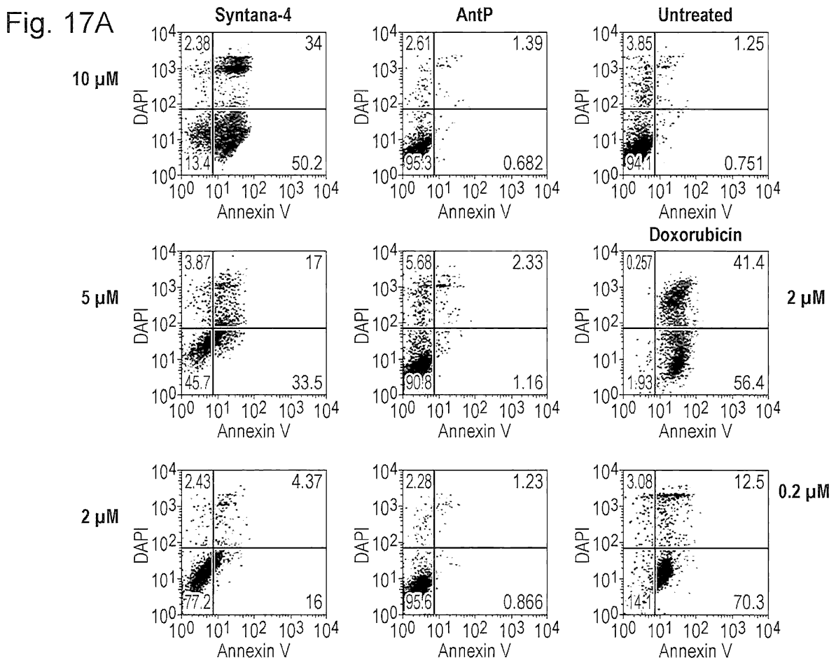

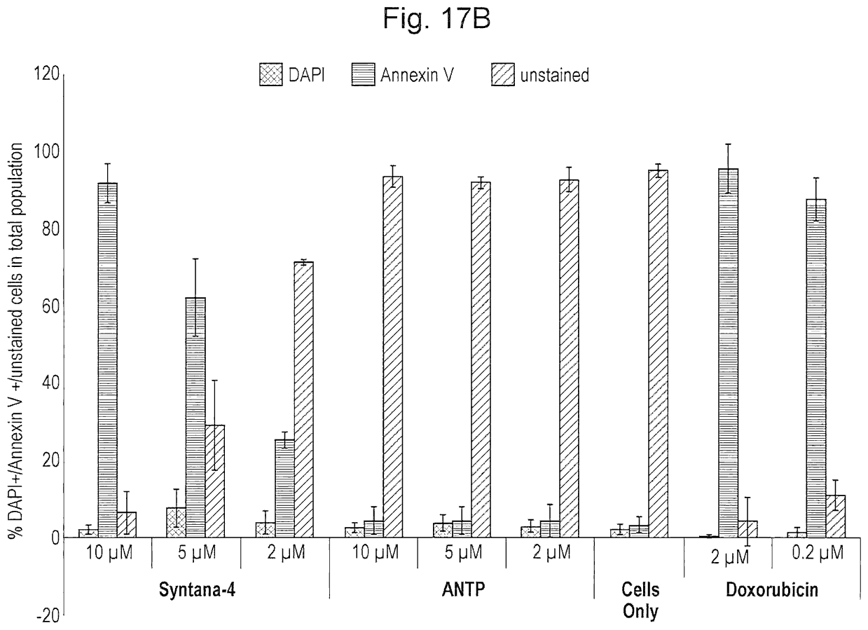

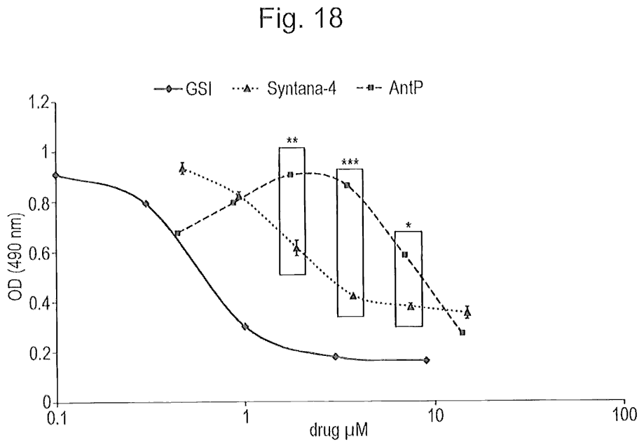

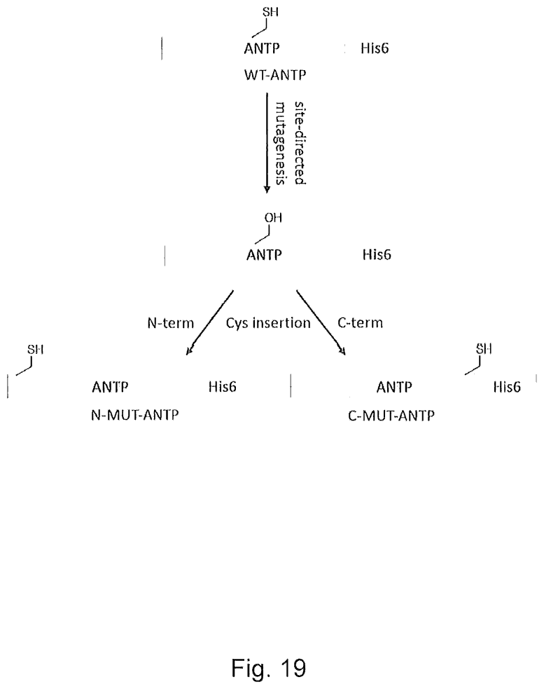

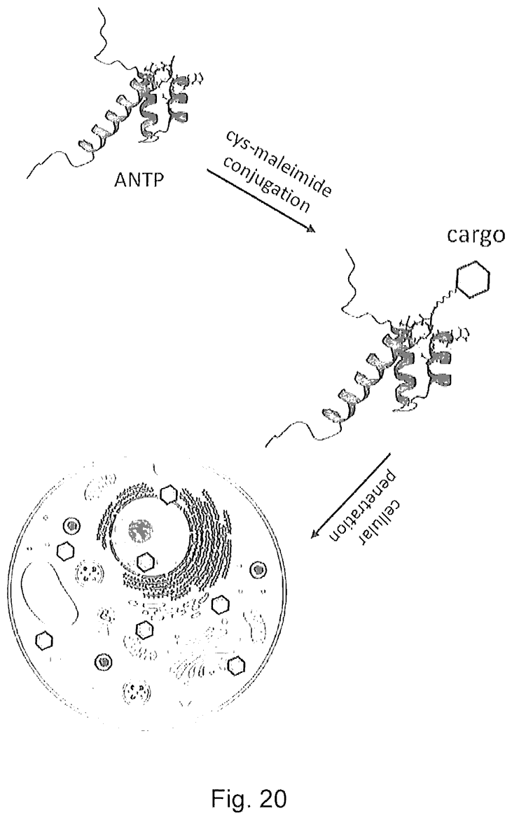

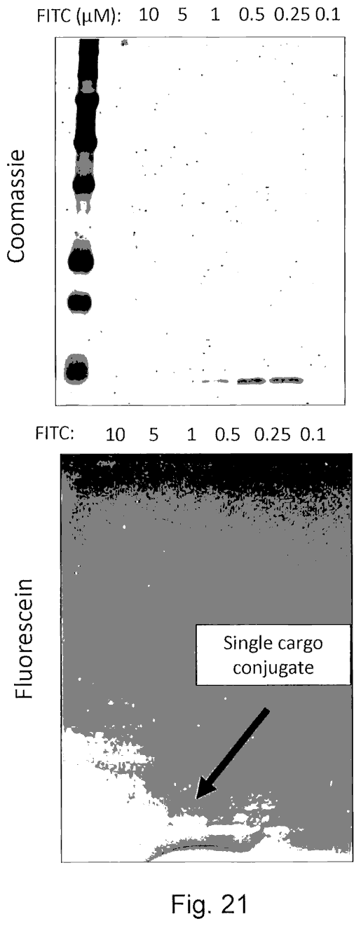

E. coli cells expressing an ANTP construct (U.S. Pat. No. 8,748,112) was lysed and the ANTP peptide was purified by either cation ion exchange (FIG. 3a) or size exclusion chromatography (FIG. 3c). According to the first method, the cell extract was applied to a cation ion exchange column in 10 mM phosphate buffer (pH 7). The ANTP peptide was gradient eluted with 1M NaCl in 10 mM phosphate buffer (pH 7). Purity was calculated as being at least 80% and yields were in excess of 10 mg/L bacterial culture. When purified using Superdex-75 size exclusion chromatography of the ion exchange, the peptide was at least 95% pure. The identity of the peptides was confirmed using anti-HIS blotting (FIG. 3b and FIG. 3d respectively). The circular dichroism spectrum of purified ANTP was measured between 190 and 270 nm. When dissolved in 10 mM sodium phosphate buffer (pH 7) at a concentration of 1 mg/ml isolated ANTP folds correctly as shown by its secondary structure (FIG. 4).

Recombinant ANTP-dnMAML(13-74) fusion proteins were produced using pET-based T7 expression vectors in either BL21(DE3), JM109(DE3) or Rosetta competent cells. Small amounts of recombinant ANTP-dnMAML peptides were expressed and formed insoluble intracellular aggregates (inclusion bodies). The inclusion bodies were extracted from bacteria using standard protocols (e.g. Triton-X100 extraction). Extracted recombinant peptides were then solubilised (e.g. using 6M GuHCl). Recombinant ANTP-dnMAML (TR4) was then purified by IMAC under denaturing conditions in 8M Urea and refolded by stepwise dialysis into PBS buffer using well-known refolding methods (FIG. 5). On average, peptide yield was at least 0.1 mg/L bacterial culture.

Attempts to produce recombinant ANTP-dnMAML peptides in other host organisms or on a larger scale were unsuccessful. It was discovered that recombinant ANTP-dnMAML was unable to correctly fold and thus associated with the membrane component of the cells leading to insolubility and cell toxicity. Recombinant expression using Pichia pastoris was also tried, but the yields were low and most of the material was insoluble. The use of mammalian expression systems was also unsuccessful. For example, CHO cells transfected with a ANTP-dnMAML construct expressed under the control of a pCMV-based promoter, failed to express ANTP-dnMAML (FIG. 6). This result may be a result of recombinant protein induced toxicity.

For the following in vivo studies, the purity of the recombinant ANTP-dnMAML TR4 was estimated to be approximately 20% by SDS-PAGE.

Breast cancer xenografts (MDA-MB-231) established in mice were used to assess the in vivo potency of TR4. The mice were divided into two groups, and injected every two days with 18 injections of either PBS as a control, or with recombinant ANTP/DN-MAML fusion protein (n=6 per group). Control mice treated with PBS developed rapidly growing tumors (FIG. 7).

The immunogenicity of recombinant ANTP/DN-MAML was investigated in immune-competent mice. Animals were immunized intravenously with recombinant ANTP/DN-MAML (0.2 ml, 2.5 mg/ml) without adjuvant, once per day for 5 days. Mice were bled once per week over a 4-month period, and the immune response monitored by ELISA. Blood samples were diluted 1:10, 1:100 and 1:1000 in PBS, and the immune response was monitored by ELISA on native recombinant ANTP/DN-MAML (coated at 50 pg/ml) and detected using anti-mouse antibodies. The results indicated that recombinant ANTP/DN-MAML does not raise an immune response in immunocompetent mice at a dose of 2.5 mg per week.

To determine the maximum tolerated dose, recombinant ANTP/DN-MAML tail vein administration was started when the mice reached an age of 12 weeks. Mice were continuously monitored for signs of hypoglycemic shock or drug side effects and were sacrificed if body weight loss exceeded 15%. Various dosages were tested starting at 4 mg/kg/day. It was found that 57 mg/kg/day of ANTP/DN-MAML is the maximum tolerated dose. At this dose, mice suffered from loss of appetite, weight loss and hypoglycemia. This experiment was terminated by sacrificing the animals three days after injection.

### Example 3: Synthetic Production of ANTP-MAML (Syntana-4)

Syntana-4 (SEQ ID NO:12) was synthesized by SPPS. Unexpectedly the process was high yielding and the peptide conjugate was functional. The full length peptide conjugate was purified by reverse-phase HPLC using an aqueous mobile phase consisting of 0.1% TFA in water, an organic mobile phase consisting of 0.1% TFA in acetonitrile, wherein the proportion of organic buffer was increased from 22-55% over 20 minutes. The eluted conjugate was at least 97% pure. The peptide was subsequently lyophilised and stored at −20° C.

The conjugate was analysed by mass spectrometry. The expected MW is 16896 and observed was 16982 (FIG. 8) indicating that the correct peptide sequence had been made.

10 mg of Syntana-4 peptide was dissolved in 7 ml tissue culture grade PBS, gently vortexed and left at 4° C. for 48 hours. This equalled 1 mg/ml of net peptide (70% peptide content). The yield of soluble peptide was greater than 95%. Samples were aliquoted and stored frozen and were kept refrigerated throughout the various experiments.

Recombinant ANTP and Syntana-4 was analysed by circular dichroism (CD) to assess the helical content. All samples gave a characteristic alpha-helix pattern. Some distortion of the CD spectra was seen at the lower wavelengths due to the high salt content, known to interfere with CD. ANTP showed the double minima typical of highly alpha-helical peptide structures in PBS buffer (FIG. 9a). When dissolved in one of 4 buffers, namely PBS (FIG. 9b), Non-buffered saline 0.9% (FIG. 9c), HEPES buffer (FIG. 9d), and 1 mM Tris-HCL pH 7.5, 5% PEG (FIG. 9e), Syntana-4 peptide regained a similarly high alpha-helical structures of 50-60%, consistent with the predicted 65% structure.

In Example 1, recombinant ANTP/DNMAML (TR4) was administered to mice as an impure formulation. Using SDS-PAGE the purity of TR4 administered to mice in Example 1 was estimated to be approximately 20%. Therefore, the concentration of TR4 used in these experiments is an overestimation. Instead, the inventors have demonstrated that TR4, prepared as described in Example 1, could not be concentrated beyond 0.5 mg/mL in PBS buffer without displaying signs of aggregation (visible precipitation).

In contrast, the purity of Syntana-4 is very high (99%). Pure Syntana-4 was also stable and soluble at 1 mg/mL and 5 mg/mL in PBS buffer as observed by its secondary structure (FIG. 10), with a characteristic alpha-helical plot which was linearly concentration dependent. Control samples, AntP and dnMAML also displayed alpha-helical structural properties. Other methods for determining relative protein concentrations and aggregation are known in the art, including liquid chromatography, multi-angled light-scattering, analytical ultracentrifugation and spectroscopic techniques.

### Example 4: Dye Conjugations with Syntana-4

The molecular structure of ANTP (generated using Swiss PDB viewer using the solved NMR structure and the data files available from the RCSB Protein Data Bank https://www.rcsb.org/pdb/explore/explore.do?structureId=1SAN) shows exposed lysine residues and one exposed cysteine residue suitable for fluorescent labelling (FIG. 11).

Commercially available fluorescent dyes (FIG. 12) were conjugated to Syntana-4. Pilot conjugations were carried out on 1 mg/ml Syntana-4 peptide samples. 100 μg of Syntana-4 peptide was dissolved in PBS and treated with 10 mM TCEP to reduce the thiols for 1 hr. The samples were desalted in Zeba columns and reacted with a 20-fold molar excess of each dye. The unreacted dye was quenched with 40-fold free cysteine and purified by Zeba desalting columns. The UV-vis spectra was used to determine quality of conjugation and peak absorbance shifts (FIG. 13).

Syntana-4-IR, Syntana-4-Cy5 and Syntana-4-Cy5.5 all fluoresced as expected. The Syntana-4 peptide-IR dye peak (arrow, 2) is at 690 nm with a smaller peak in the required region of 620 nm. The peaks were sharp indicating a soluble conjugate, but the peptide peak (280 nm) was less sharp. The Cy5 conjugate peaks were at 600 nm and 650 nm (arrow, 2). The peaks were sharp indicating a soluble conjugate but the peptide peak (arrow, 1, 280 nm) was less sharp. The Cy5.5 conjugate peaks were at 630 nm and 680 nm (arrow, 2). The peaks were sharp indicating a soluble conjugate and the peptide peak (arrow, 1, 280 nm) was more sharp than the other two dye conjugates.

SDS PAGE gels viewed under fluorescence showed that the Syntana-4 peptide-IR dye conjugate was the brightest but had side-reaction products. The Cy5.5 and Cy5 conjugates were cleaner and showed fluorescent properties.

Syntana-4 can be successful conjugated to maleimide-based dyes (FIG. 13) showing that the Syntana-4 free thiol was accessible. Syntana-4-IR, Syntana-4-Cy5 and Syntana-4-Cy5.5 fluoresce as expected.

### Example 5: Tumour Growth Inhibition

16 BALB/c nude mice (6-8 weeks old) were inoculated with 2 million MDA-MB231 tumour cells in ice-cold 50% DMEM media/FCS+50% matrigel, subcutaneously. These tumours were monitored and used when they had grown to around 24-100 mm³ (around 4-5 mm diameter). The mice were randomised and grouped (6 in Syntana-4 therapy, 5 in chemotherapy and 5 saline treated). The 16 mice were treated as follows:

| Group | Sample size | Therapy | Dosage regime |
|---|---|---|---|
| 1 | 6 | Syntana-4 therapy | 4 mg/kg, 3 times |
| | | | per week, 8 |
| | | | doses (approx 0.1 |
| | | | mg/mouse) |
| 2 | 5 | Control-chemotherapy | 10 mg/kg, 2 times |
| | | Paclitaxel (PTX) | per week, 5 doses |
| 3 | 5 | Control-saline treated | 3 times per week, |
| | | | 8 doses |

At the end of the treatment regime, the animals were culled and dissected. The GI tract was removed and washed through with sterile saline solution. The tumours were dissected and divided in two. Half of the tumour was snap frozen in liquid nitrogen and used to make mRNA for Q-PCR of Notch genes. The other half of the tumour was paraffin-embedded and sectioned (5-10 μm) onto slides. 4 Syntana-4 treated tumours produced satisfactory tissue pieces for evaluation.

Tumour sizes were calculated as (L×W×W)/2 and plotted as a percentage change from the day treatment started (FIG. 14). In two independent experiments, Syntana-4 was able to delay tumour growth compared to standard chemotherapy (Paclitaxel). Using ANOVA, which takes into account the growth progress (repeated measures), the reduced tumour growth compared to the controls (Paclitaxel and Saline) is statistically significant.

The Syntana-4 data points for days 14, 16 and 18 are (Students T-test)

| Comparison | Day 14 P-value | Day 16 P-value | Day 18 P-value |
|---|---|---|---|
| Saline vs Syntana-4 | 0.01, significant | 0.04, significant | 0.03, significant |
| Chemotherapy vs | 0.14. not | 0.12, not | 0.04, Significant |
| Syntana-4 | significant | significant | |

The P-values for the significance of responses (2-way ANOVA) are

| Comparison | P-value | Significant difference? |
|---|---|---|
| Saline vs Syntana-4 | 0.006 | YES |
| Chemotherapy vs Syntana-4 | 0.11 | NO |

### Example 6: Pharmacodynamic Studies

RT-Quantitative-PCR was performed to assess the effect of Syntana-4 on the expression on Notch target genes and Notch-1 and Notch-4 genes. mRNA was extracted from snap frozen excised tumour tissue using the RNAEasy QIAGEN kit. cDNA was produces from 0.5 μg of total RNA, using the Roche First Strand DNA synthesis kit. The table below summarises fold changes in gene expression from four Syntana-4 treated tumours compared to control (saline treated) animals. The gene expression levels were also normalised using the internal GAPDH standard (FIG. 15).

Assessment of HES5 and HEY2 can be used to provide a robust pharmacodynamic readout of Syntana-4 activity in tumour tissue. This provides evidence for target gene transcriptional inhibition. Gene expression analysis showed that HES5 and HEY2 genes are consistently down-regulated in MDA-MB231 xenograft tumours treated with Syntana-4. There is a variable effect on other tested genes. Hes-5 seems to be more affected (6-fold to 20-fold reduction in mRNA expression) than Hes-2 (up to 3-fold).

### Example 7: Immuno-Histochemistry of Syntana-Treated Tumours

Immuno-histochemistry staining was performed using a Ki67 antibody assay to evaluate the effect of Syntana-4 on the proliferative capacity of tumour cells (FIG. 16). Tumours were exposed to either Syntana-4 or saline according to the table below.

| | | | |
|---|---|---|---|
| 1-1 | Syntana-4 treated | Mouse 2 | no reduction |
| 1-2 | Syntana-4 treated | Mouse 2 | significant reduction |
| 1-2(2) | Syntana-4 treated | Mouse 2 (area 2) | no significant reduction |
| 1-3 | Syntana-4 treated | Mouse 3 | no significant reduction |
| 1-4 | Syntana-4 treated | Mouse 4 | significant reduction |
| 1-4 | Syntana-4 treated | Mouse 4 (area 2) | no significant reduction |
| 1-6 | Syntana-4 treated | Mouse 6 | no significant reduction |
| 2-1 | Saline treated | Mouse 1 | no reduction |
| 2-2 | Saline treated | Mouse 1 | no reduction |

Ki67 staining is positive in greater than 80% of cells in control tumours (arrows). This is expected for an MDA-MB-231 xenograft model. There is a moderate but significant reduction in tumours 1-2, and 1-4, where between 40-60% of cells show positive Ki67 staining (bold arrows). Therefore, there is significant reduction in cellular proliferation in areas of tumours treated with Syntana-4 compared to no reduction in any areas treated with a saline control.

### Example 8: Syntana-4 Causes Apoptosis of MDA-MB-231 Cells

Apoptotic cells were identified and quantified by Annexin V-DAPI staining. Cells were plated at 15,000 cells/well and treated 48 h later in triplicate with Syntana-4, ANTP or doxorubicin. After 72 h, cells were analysed by flow cytometry. Syntana-4, as expected from the mechanism of action, causes increased apoptosis (FIG. 17a, top right quandrant). Apoptotic cells as a percentage of the total number of collected cells was calculated from the histograms.

### Example 9: Syntana-4 Causes Growth Inhibition MDA-MB-231 Cells

Following treatment of MDA-MB-231 cells with either Syntana-4, GSI-1 inhibitor or ANTP, cell proliferation was quantified. Cells were plated at 5000 cells/well and treated 48 h later. Test agents were exposed for 72 h and cell proliferation measured by Cell Titre-96 assay (Promega). Each point is a mean±SD. The carrier solution of 1% DMSO (for GSI-1) had no effect on the cells (Abs=0.85). Untreated control Abs=0.89. One-way ANOVA was used for statistical comparison. Proliferation, was shown to be significantly inhibited in Syntana-4 treated cells using a one-way ANOVA test (FIG. 18).

### Example 10: Expression, Purification and Chemical Ligation of Two Components to Form ANTP-Thiol

A single E. coli BL21 (DE3) plysS colony transformed with ANTP-thiol gene in pET23b was grown in 500 ml of LB media containing carbenicillin and chloramphenicol and grown shaking at 37° C. up to an OD600 of 1.2. The culture was then induced using 1 mM IPTG for 5 hours. The culture was centrifuged at 2000 g for 30 minutes. The cell pellet was freeze-thawed and resuspended in 20 ml of 50 mM sodium phosphate (pH 7.5), 2 mM EDTA, 1 mM DTE with the inclusion of a protease inhibitor tablet (Roche). Sonication was used to further lyse the cells before centrifuging at 25,000 g for 30 minutes at 4° C. The soluble fraction was applied to a pre-equilibrated SP sepharose fast flow (GE healthcare) ion exchange column (5 ml slurry). ANTP-thiol was washed and eluted using increasing concentrations of NaCl (50 mM-1M) (25 ml each). The fractions containing ANTP protein were pooled and concentrated to 5 mg/mL using a 3,000 kDa MWCO spin concentrator (Vivaspin, Generon) before being applied to a size exclusion chromatography column (Superdex-75) and eluted in PBS. The absorbance of fractions was measured at 214 nm and 280 nm using the QuadTec detector in combination with the Bio-Rad BioLogic Duo-Flow™ system. The collected fractions were analysed for ANTP-thiol protein and pooled and concentrated to 1 mg/mL.

A single E. coli BL21 (DE3) plysS colony transformed with MAML gene bearing a HIS-Tag in pET23b was grown in 500 ml of LB media containing carbenicillin and chloramphenicol and grown shaking at 37° C. up to an OD600 of 1.2. The culture was then induced using 1 mM IPTG for 16 hours. The culture was centrifuged at 2000 g for 30 minutes. The cell pellet was freeze-thawed and resuspended in 20 ml of 50 mM sodium phosphate (pH 7.5), 2 mM EDTA, 1 mM DTE with the inclusion of a protease inhibitor tablet (Roche). Sonication was used to further lyse the cells before centrifuging at 25,000 g for 30 minutes at 4° C. The soluble fraction was applied to a pre-equilibrated IMAC Talon column (Sigma), and purified according to the manufacturer's instructions. MAML was washed and eluted using increasing concentrations of Imidazole (50 mM-1M) (25 ml each). The fractions containing MAML protein were pooled and concentrated to 5 mg/mL using a 3,000 kDa MWCO spin concentrator (Vivaspin, Generon) before being applied to a size exclusion chromatography column (Superdex-75) and eluted in PBS. The absorbance of fractions was measured at 214 nm and 280 nm using the QuadTec detector in combination with the Bio-Rad BioLogic Duo-Flow™ system. The collected fractions were analysed for MAML protein and pooled and concentrated to 1 mg/mL.

1 mL of 2 mg/mL ANTP-thiol, dissolved in PBS with 1 mM EDTA was reduced with 5 molar equivalents of TCEP for 2 h at room temperature. The reduced ANTP-thiol was reacted with 1 mL of 2 mg/mL MAML dissolved in sodium bicarbonate, 1 mM EDTA 100 mM NaCl, pH 9.2 was reacted with 5 molar equivalents of a cross linker ‘maleimide-PEG-2-succinimidyl ester for 2 h at room temperature. Unreacted cross linker and multivalent MAML was removed by size-exclusion chromatography to yield MAML with a single maleimide cross-linker per MAML protein. This was mixed with an equimolar amount of reduced ANTP-thiol to allow the maleimide to react with the thiol to form cross-linked ANTP-PEG-2-MAML, linked by a PEG-2 containing linker and thioether bond.

### Example 11: Cell Transduction with an Improved ANTP-PEG-2-MAML

Human cell-line SKOV3 was cultured in DMEM media with 10% serum at 37° C., 5% CO₂ until 40% confluency in a 96-well culture dish. The media was removed and replaced with 0.1 mL tissue culture-grade PBS and unmodified ANTP, unmodified MAML and ANTP-PEG-2-MAML were added to a final concentration of 0.01 mg/mL. Cell transduction was allowed to proceed for 1 hour at 37° C. Next, the PBS was removed and replaced with DMEM/serum and the cells were grown overnight as above. The transduced cells were then fixed using 1% glutaraldehyde at room temperature for 1 hr and permeabilized using Saponin at 1% concentration for 20 minutes at room temperature. Cells were then washed with PBS and visualized using FITC-conjugated-anti-HIS antibody (Miltenyi Biotech). Imaging was done using a GE-Incell-500 analyser visualizing the green fluorescence of the FITC at around 525 nm. The MAML-His protein could be seen inside the cell cytosol and cell nucleus of the ANTP-PEG-2-MAML transduced cells, but not the ANTP or MAML only treated cells. This confirms the functionality of the ANTP-PEG-2-MAML protein.

### Example 12: ANTP Expression and Conjugation with FITC-Maleimide

E. coli BL21 were transformed with wild type ANTP, as described in Example 10. Expression of ANTP was induced by IPTG induction. Wild type ANTP was bound to Ni-NTA resin in binding buffer. The resin was washed with wash buffer and PBS. FITC-maleimide conjugation was performed on the resin using 50 μM FITC-Mal solution in 1×PBS for 1 hr at room temperature. The controls did not comprise FITC-maleimide. The proteins were eluted with elution buffer (PBS, 200 mM imidazole). The inventors screened different concentrations and conditions for on resin ligation to wild type ANTP. The dominant conjugated species was to Cys-39 of ANTP (see, the schematic in FIG. 20 and the blots in FIG. 21). Selectivity for Cyc-39 is high. However, the inventors considered that accessibility for conjugation could be improved by using alternative ANTP derivatives (see, Example 13).

### Example 13: Site Directed Mutagenesis of ANTP

The inventors modified the wild type ANTP sequence by site-directed mutagenesis to produce variant ANTP and ANTP-dnMAML sequences with advantageous properties. In particular, the wild type ANTP sequence (SEQ ID NO:2) was modified according to the schematic of FIG. 19. Site directed mutagenesis was used to replace the internal cysteine with a serine (C39S) and to add either an N or C terminal cysteine.

Exemplary improved ANTP proteins (ANTP-thiol) include the amino acid sequences as follows (mutations shown in bold/underline):

| ID Sequence | |
|---|---|
| 13 | RKRGRQTYTRYQTLELEKEFHFNRYLTRRRRIEIAHALSLTERQIKIWFQNRRM |
| | KWKKENC |
| 14 | CRKRGRQTYTRYQTLELEKEFHFNRYLTRRRRIEIAHALSLTERQIKIWFQNR |
| | RMKWKKEN |
| 15 | CRKRGRQTYTRYQTLELEKEFHFNRYLTRRRRIEIAHALSLTERQIKIWFQNR |
| | RMKWKKENC |
| 16 | RKRGRQTYTRYQTLELEKEFHFNRYLTRRRRIEIAHALSLTERQIKIWFQNRR |
| | MKWKKEN |

Replacement of Cys-39 with serine is considered to allow for maintaining or improving the membrane translocation or stability properties of ANTP whilst reducing the presence of thiol groups. Introduction of thiol residue(s) through mutagenesis at the N and C terminal positions allowed to produced useful constructs for chemical conjugation of a cargo molecule (e.g., dnMAML) using thiol-maleimide chemistry (compare to the schematic in FIG. 20).

### Embodiments of the Invention

1. A cell-penetrating peptide (CPP) comprising from 10 to 60 contiguous amino acids selected from an Antennapedia (ANTP) protein.
2. The cell-penetrating peptide (CPP) according to embodiment 1, comprising from 10 to 60 contiguous amino acids selected from the sequence of SEQ ID NO: 2, or a variant thereof having at least 80% sequence identity to a sequence of from 10 to 60 contiguous amino acids selected from the sequence of SEQ ID NO: 2.
3. The cell-penetrating peptide (CPP) according to embodiment 2, having the sequence of SEQ ID NO: 4 or a sequence having at least 80% sequence identity to SEQ ID NO: 4.
4. The cell-penetrating peptide (CPP) according to embodiment 2 or 3, wherein the stability and/or transduction efficiency are improved compared to the stability and/or transduction efficiency of a CPP consisting of the sequence of SEQ ID NO: 2 and/or SEQ ID NO: 4.
5. The cell-penetrating peptide (CPP) according to any one of embodiments 1 to 4, wherein the immunogenicity of the CPP is reduced compared to the immunogenicity of a CPP consisting of the sequence of SEQ ID NO: 2 and/or SEQ ID NO: 4.
6. The cell-penetrating peptide (CPP) according to any one of the preceding embodiments, wherein, compared to the sequence of SEQ ID NO: 2, the CPP variant comprises a mutation of any arginine to lysine.
7. The cell-penetrating peptide (CPP) according to any one of the preceding embodiments, wherein, compared to the sequence of SEQ ID NO: 2, the CPP variant comprises a mutation of any lysine to arginine or another tolerated residue.
8. The cell-penetrating peptide (CPP) according to any one of the preceding embodiments, wherein, compared to the sequence of SEQ ID NO: 2, the CPP variant comprises replacement of Cys-39 with any tolerated residue.
9. The cell-penetrating peptide (CPP) according to any one of the preceding embodiments, wherein, compared to the sequence of SEQ ID NO: 2, the CPP variant comprises one or more alternative thiol residues at positions 1 to 60.
10. The cell-penetrating peptide (CPP) according to any one of the preceding embodiments, wherein, compared to the sequence of SEQ ID NO: 2, the CPP variant comprises mutation of one or more non-basic residue into a basic residue.
11. The cell-penetrating peptide (CPP) according to any one of the preceding embodiments, wherein, compared to the sequence of SEQ ID NO: 2, the CPP variant comprises the introduction of any stabilising mutations, such as disulphide bridges, ionic interactions, hydrophobic interactions, reduced proteolysis liabilities.
12. The cell-penetrating peptide (CPP) according to any one of the preceding embodiments, wherein, compared to the sequence of SEQ ID NO: 2, the CPP variant comprises chemical modification of any residue 1-60, such as alkylation, cross-linking, or stapling.
13. The cell-penetrating peptide (CPP) according to any one of the preceding embodiments, wherein, compared to the sequence of SEQ ID NO: 2, the CPP variant comprises the addition of any chemical modification at the N-terminus or C-terminus; optionally, wherein the modification improves the chemical conjugation, membrane translocation or stability properties of the CPP.
14. The cell-penetrating peptide (CPP) according to any one of the preceding embodiments, wherein, compared to the sequence of SEQ ID NO: 2, the CPP variant comprises the replacement of any natural amino acid (L-amino acid) with non-natural amino acids (D-amino acid).
15. A fusion protein comprising the cell-penetrating peptide (CPP) as defined in any one of embodiments 1 to 14 and a therapeutically useful protein.
16. The fusion protein according to embodiment 15 wherein the therapeutically useful protein is a dominant-negative protein; optionally wherein the dominant-negative protein is a dominant-negative mastermind-like protein (DN-MAML), AKT-DN or STAT3; further optionally wherein the DN-MAML has the sequence of SEQ ID NO: 9 or a sequence with 80% sequence identity thereto.
17. The fusion protein according to any one of embodiments 15 or 16, wherein the cell-penetrating peptide (CPP) is covalently linked to the dominant-negative protein;
   optionally wherein the covalent link is a peptide bond, a chemical bond, further optionally wherein the covalent link is a chemical bond using chemically-modified amino acid and/or wherein the cell-penetrating peptide (CPP) is covalently linked to the dominant-negative protein by a linker peptide.
18. The fusion protein according to embodiment 17, wherein the cell-penetrating peptide (CPP) is covalently linked to the dominant-negative protein by a zero-length crosslinker, a homobifunctional crosslinker, a hetero-bifunctional crosslinker or a trifunctional crosslinkers; optionally wherein
the zero-length crosslinker is selected from carbodiimides (EDC, EDC plus Sulfo-NHS, CMC, DCC, DIC), Woodward's Reagent K, N,N-Carbonyldiimidazole, Schiff Base Formation and Reductive Amination);
the homobifunctional crosslinkers is selected from Homobifunctional NHS Esters (DSP and DTSSP, DSS and BS3, DST and Sulfo-DST, BSOCOES and Sulfo-BSOCOES, EGS and Sulfo-EGS, DSG, DSC), Homobifunctional Imidoesters (DMA, DMP, DMS, DTBP), Homobifunctional Sulfhydryl-Reactive Crosslinkers (DPDPB, BMH), Difluorobenzene Derivatives (DFDNB, DFDNPS), Homobifunctional Photoreactive Crosslinkers (BASED), Homobifunctional Aldehydes (Formaldehyde, Glutaraldehyde), Bis-epoxides (1,4-Butanediol Diglycidyl Ether), Homobifunctional Hydrazides, Adipic Acid, Dihydrazide, Carbohydrazide), Bis-diazonium Derivatives (o-Tolidine, Diazotized, Bis-diazotized Benzidine), Bis-alkyl Halides;
the hetero-bifunctional Crosslinker is selected from Amine-Reactive and Sulfhydryl-Reactive Crosslinkers (SPDP, LC-SPDP, and Sulfo-LC-SPDP, SMPT and Sulfo-LC-SMPT, SMCC and Sulfo-SMCC, MBS and Sulfo-MBS, SIAB and Sulfo-SIAB, SMPB and Sulfo-SMPB, GMBS and Sulfo-GMBS, SIAX and SIAXX, SIAC and SIACX, NPIA), Carbonyl-Reactive and Sulfhydryl-Reactive Crosslinkers (MPBH, M2C2H, PDPH), Amine-Reactive and Photoreactive Crosslinkers (NHS-ASA, Sulfo-NHS-ASA, and Sulfo-NHS-LC-ASA, SASD, HSAB and Sulfo-HSAB, SANPAH and Sulfo-SANPAH, ANB-NOS, SAND, SADP and Sulfo-SADP, Sulfo-SAPB, SAED, Sulfo-SAMCA, p-Nitrophenyl Diazopyruvate, PNP-DTP), Sulfhydryl-Reactive and Photoreactive Crosslinkers (ASIB, APDP, Benzophenone-4-iodoacetamide, Benzophenone-4-maleimide), Carbonyl-Reactive and Photoreactive Crosslinkers (ABH), Carboxylate-Reactive and Photoreactive Crosslinkers (ASBA), Arginine-Reactive and Photoreactive Crosslinkers (APG); and
the trifunctional crosslinker is selected from 4-Azido-2-nitrophenylbiocytin-4-nitrophenyl ester, Sulfo-SBED, MTS-ATF-Biotin and MTS-ATF-LC-Biotin, Hydroxymethyl Phosphine Derivatives.
19. A pharmaceutical composition comprising the cell penetrating peptide as defined in any one of embodiments 1 to 14 or the fusion protein as defined in any one of embodiments 15 to 18.
20. The cell penetrating peptide as defined in any one of embodiments 1 to 14 or the fusion protein as defined in any one of embodiments 15 to 18 or the pharmaceutical composition as defined in embodiment 19 for use in a method of therapy.
21. The cell penetrating peptide as defined in any one of embodiments 1 to 14 or the fusion protein as defined in any one of embodiments 15 to 18 or the pharmaceutical composition as defined in embodiment 19 for use in method of treating cancer.
22. The cell penetrating peptide, the fusion protein or the pharmaceutical composition for use as defined in embodiment 21, wherein administration of the cell penetrating peptide, fusion protein or the pharmaceutical composition inhibits Notch signalling in a cancer cell.
23. A method of treating cancer, the method comprising the administration of a cell penetrating peptide as defined in any one of embodiments 1 to 14 or the fusion protein as defined in any one of embodiments 15 to 18 or the pharmaceutical composition as defined in embodiment 19 to a subject in need thereof.
24. Use of the cell penetrating peptide as defined in any one of embodiments 1 to 14 or the fusion protein as defined in any one of embodiments 15 to 18 or the pharmaceutical composition as defined in embodiment 19 in the manufacture of a medicament for the treatment of cancer.
25. A method for producing a fusion protein, the method comprising linking the cell-penetrating peptide (CPP) as defined in any one of embodiments 1 to 14 and a therapeutically useful protein by any of the following methods: activated-esters coupling of lysine residues; methanesulfonyl acrylate coupling of lysine residues; copper and non-catalysed ‘click’ reactions of alkynes introduced into proteins; disulphide bonds formed with native or engineered cysteine thiol groups; thioether bonds formed with cysteine thiols and introduced maleimide groups; native chemical ligation; smartag chemo-selective ligation using formyl-glycine modified proteins; chemoselective azo-coupling reactions; cyclo-addition reactions (Cu-AAC or SPAAC) of azide-derivatized amino acids; and/or photo-reactive or photo-catalysed chemical reactions.

### REFERENCES

Arai, R., Wriggers, W., Nishikawa, Y., Nagamune, T. and Fujisawa, T., 2004. Conformations of variably linked chimeric proteins evaluated by synchrotron X-ray small-angle scattering. PROTEINS: Structure, Function, and Bioinformatics, 57(4), pp. 829-838.
Bitler, B. G., Menzl, I., Huerta, C. L., Sands, B., Knowlton, W., Chang, A. and Schroeder, J. A., 2009. Intracellular MUC1 peptides inhibit cancer progression. Clinical Cancer Research, 15(1), pp. 100-109.
Bloch-Gallego, E., Le Roux, I., Joliot, A. H., Volovitch, M., Henderson, C. E. and Prochiantz, A., 1993. Antennapedia homeobox peptide enhances growth and branching of embryonic chicken motoneurons in vitro. The Journal of cell biology, 120(2), pp. 485-492.
Bolhassani, A., 2011. Potential efficacy of cell-penetrating peptides for nucleic acid and drug delivery in Cancer. Biochimica et Biophysica Acta (BBA)-Reviews on Cancer, 1816(2), pp. 232-246.
Cancer Genome Atlas Research Network, 2013. Comprehensive molecular characterization of clear cell renal cell carcinoma. Nature, 499(7456), pp. 43-49.
Chaloin, L., Vidal, P., Heitz, A., Van Mau, N., Méry, J., Divita, G. and Heitz, F., 1997. Conformations of primary amphipathic carrier peptides in membrane mimicking environments. Biochemistry, 36(37), pp. 11179-11187.
Chen X, Zaro J L, Shen W C. Fusion protein linkers: property, design and functionality. Adv Drug Deliv Rev. 2013 October; 65(10):1357-69. doi: 10.1016/j.addr.2012.09.039. Epub 2012 Sep. 29. PMID: 23026637; PMCID: PMC3726540.
Cronican, J. J., Beier, K. T., Davis, T. N., Tseng, J. C., Li, W., Thompson, D. B., Shih, A. F., May, E. M., Cepko, C. L., Kung, A. L. and Zhou, Q., 2011. A class of human proteins that deliver functional proteins into mammalian cells in vitro and in vivo. Chemistry & biology, 18(7), pp. 833-838.
De Strooper, B., Annaert, W., Cupers, P., Saftig, P., Craessaerts, K., Mumm, J. S., Schroeter, E. H., Schrijvers, V., Wolfe, M. S., Ray, W. J. and Goate, A., 1999. A presenilin-1-dependent γ-secretase-like protease mediates release of Notch intracellular domain. Nature, 398(6727), pp. 518-522.
Duchardt, F., Fotin-Mleczek, M., Schwarz, H., Fischer, R. and Brock, R., 2007. A comprehensive model for the cellular uptake of cationic cell-penetrating peptides. Traffic, 8(7), pp. 848-866.
Erazo-Oliveras, A., Muthukrishnan, N., Baker, R., Wang, T. Y. and Pellois, J. P., 2012. Improving the endosomal escape of cell-penetrating peptides and their cargos: strategies and challenges. Pharmaceuticals, 5(11), pp. 1177-1209.
Herskowitz, I., 1987. Functional inactivation of genes by dominant negative mutations. Nature, 329(6136), pp. 219-222.
Huang, Q. B., Ma, X., Li, H. Z., Ai, Q., Liu, S. W., Zhang, Y., Gao, Y., Fan, Y., Ni, D., Wang, B. J. and Zhang, X., 2014. Endothelial Delta-like 4 (DLL4) promotes renal cell carcinoma hematogenous metastasis. Oncotarget, 5(10), p. 3066
Jarriault, S., Brou, C., Logeat, F., Schroeter, E. H., Kopan, R. and Israel, A., 1995. Signalling downstream of activated mammalian Notch. Nature, 377(6547), pp. 355-358.
Jones, A. T. and Sayers, E. J., 2012. Cell entry of cell penetrating peptides: tales of tails wagging dogs. Journal of Controlled Release, 161(2), pp. 582-591.
Lee, M., Kim, H. J., Kim, S. W., Park, S. A., Chun, K. H., Cho, N. H., Song, Y. S. and Kim, Y. T., 2016. The long non-coding RNA HOTAIR increases tumour growth and invasion in cervical cancer by targeting the Notch pathway. Oncotarget, 7(28), p. 44558.
Kousparou, C. A., Yiacoumi, E., Deonarain, M. P. and Epenetos, A. A., 2012. Generation of a selectively cytotoxic fusion protein against p53 mutated cancers. BMC cancer, 12(1), p. 338.
Omholt, S. W., Plahte, E., Øyehaug, L. and Xiang, K., 2000. Gene regulatory networks generating the phenomena of additivity, dominance and epistasis. Genetics, 155(2), pp. 969-980.
Opacak-Bernardi, T., Ryu, J. S. and Raucher, D., 2017. Effects of cell penetrating Notch inhibitory peptide conjugated to elastin-like polypeptide on glioblastoma cells. Journal of drug targeting, 25(6), pp. 523-531.
Osman, G., Rodriguez, J., Chan, S. Y., Chisholm, J., Duncan, G., Kim, N., Tatler, A. L., Shakesheff, K. M., Hanes, J., Suk, J. S. and Dixon, J. E., 2018. PEGylated enhanced cell penetrating peptide nanoparticles for lung gene therapy. Journal of controlled release, 285, pp. 35-45.
Rizzuti, M., Nizzardo, M., Zanetta, C., Ramirez, A. and Corti, S., 2015. Therapeutic applications of the cell-penetrating HIV-1 Tat peptide. Drug discovery today, 20(1), pp. 76-85.
Soomets, U., Lindgren, M., Gallet, X., Hällbrink, M., Elmquist, A., Balaspiri, L., Zorko, M., Pooga, M., Brasseur, R. and Langel, Ü., 2000. Deletion analogues of transportan. Biochimica et Biophysica Acta (BBA)-Biomembranes, 1467(1), pp. 165-176.
Soudy, R., Chen, C. and Kaur, K., 2016. Correction to Novel Peptide-Doxorubucin Conjugates for Targeting Breast Cancer Cells Including the Multidrug Resistant Cells. Journal of Medicinal Chemistry, 59(7), pp. 3575-3575.
Stylianou, S., Clarke, R. B. and Brennan, K., 2006. Aberrant activation of notch signaling in human breast cancer. Cancer research, 66(3), pp. 1517-1525.
Szabó, I., Orbán, E., Schlosser, G., Hudecz, F. and Bánóczi, Z., 2016. Cell-penetrating conjugates of pentaglutamylated methotrexate as potential anticancer drugs against resistant tumor cells. European journal of medicinal chemistry, 115, pp. 361-368.
Tamura, K., Taniguchi, Y., Minoguchi, S., Sakai, T., Tun, T., Furukawa, T. and Honjo, T., 1995. Physical interaction between a novel domain of the receptor Notch and the transcription factor RBP-Jκ/Su (H). Current Biology, 5(12), pp. 1416-1423.
Vasconcelos, L., Pärn, K. and Langel, Ü., 2013. Therapeutic potential of cell-penetrating peptides. Therapeutic delivery, 4(5), pp. 573-591.
Wadia, J. S., Stan, R. V. and Dowdy, S. F., 2004. Transducible TAT-HA fusogenic peptide enhances escape of TAT-fusion proteins after lipid raft macropinocytosis. Nature medicine, 10(3), pp. 310-315.
Waldo, G. S., Standish, B. M., Berendzen, J. and Terwilliger, T. C., 1999. Rapid protein-folding assay using green fluorescent protein. Nature biotechnology, 17(7), pp. 691-695.
Wu, L., Aster, J. C., Blacklow, S. C., Lake, R., Artavanis-Tsakonas, S. and Griffin, J. D., 2000. MAML1, a human homologue of Drosophila mastermind, is a transcriptional co-activator for NOTCH receptors. Nature genetics, 26(4), pp. 484-489.
Wu, L., Sun, T., Kobayashi, K., Gao, P. and Griffin, J. D., 2002. Identification of a family of mastermind-like transcriptional coactivators for mammalian notch receptors. Molecular and cellular biology, 22(21), pp. 7688-7700.
Yu, M., Li, X., Huang, X., Zhang, J., Zhang, Y. and Wang, H., 2018. New cell-penetrating peptide (KRP) with multiple physicochemical properties endows doxorubicin with tumor targeting and improves its therapeutic index. ACS applied materials & interfaces, 11(2), pp. 2448-2458.
Zhang, D., Wang, J. and Xu, D., 2016. Cell-penetrating peptides as noninvasive transmembrane vectors for the development of novel multifunctional drug-delivery systems. Journal of Controlled Release, 229, pp. 130-139.
Zhang, L., Sha, J., Yang, G., Huang, X., Bo, J. and Huang, Y., 2017. Activation of Notch pathway is linked with epithelial-mesenchymal transition in prostate cancer cells. Cell Cycle, 16(10), pp. 999-1007.
Zhang, P., Cheetham, A. G., Lock, L. L. and Cui, H., 2013. Cellular uptake and cytotoxicity of drug-peptide conjugates regulated by conjugation site. Bioconjugate chemistry, 24(4), pp. 604-613.

1. A cell-penetrating peptide (CPP) comprising from 10 to 60 contiguous amino acids selected from an Antennapedia (ANTP) protein.

2. The cell-penetrating peptide (CPP) according to claim 1, comprising from 10 to 60 contiguous amino acids selected from the sequence of SEQ ID NO: 2, or a variant thereof having at least 80% sequence identity to a sequence of from 10 to 60 contiguous amino acids selected from the sequence of SEQ ID NO: 2.

3. The cell-penetrating peptide (CPP) according to claim 2, comprising SEQ ID NO: 4 or a sequence having at least 80% sequence identity to SEQ ID NO: 4.

4. The cell-penetrating peptide (CPP) according to claim 2 or 3, wherein the stability and/or transduction efficiency are improved compared to the stability and/or transduction efficiency of a CPP consisting of the sequence of SEQ ID NO: 2 and/or SEQ ID NO: 4.

5. The cell-penetrating peptide (CPP) according to any one of claims 1 to 4, wherein the immunogenicity of the CPP is reduced compared to the immunogenicity of a CPP consisting of the sequence of SEQ ID NO: 2 and/or SEQ ID NO: 4.

6. The cell-penetrating peptide (CPP) according to any one of the preceding claims, wherein, compared to the sequence of SEQ ID NO: 2, the CPP variant comprises a mutation of any arginine to lysine or another tolerated residue.

7. The cell-penetrating peptide (CPP) according to any one of the preceding claims, wherein, compared to the sequence of SEQ ID NO: 2, the CPP variant comprises one or more alternative thiol residues at positions 1 to 60.

8. The cell-penetrating peptide (CPP) according to any one of the preceding claims, wherein, compared to the sequence of SEQ ID NO: 2, the CPP variant comprises mutation of one or more non-basic residue into a basic residue.

9. The cell-penetrating peptide (CPP) according to any one of the preceding claims, wherein, compared to the sequence of SEQ ID NO: 2, the CPP variant comprises the introduction of any stabilising mutations, such as disulphide bridges, ionic interactions, hydrophobic interactions, or reduced proteolysis liabilities.

10. The cell-penetrating peptide (CPP) according to any one of the preceding claims, wherein, compared to the sequence of SEQ ID NO: 2, the CPP variant comprises chemical modification of any residue 1-60, such as alkylation, cross-linking, or stapling.

11. The cell-penetrating peptide (CPP) according to any one of the preceding claims, wherein, compared to the sequence of SEQ ID NO: 2, the CPP variant comprises the addition of any chemical modification at the N-terminus or C-terminus; optionally, wherein the modification improves the chemical conjugation, membrane translocation or stability properties of the CPP.

12. The cell-penetrating peptide (CPP) according to any one of the preceding claims, wherein, compared to the sequence of SEQ ID NO: 2, the CPP variant comprises the replacement of any natural amino acid (L-amino acid) with non-natural amino acids (D-amino acid).

13. The cell-penetrating peptide (CPP) according to any one of the preceding claims, wherein, the residue corresponding to Cys-39 in SEQ ID NO: 2 is replaced with any tolerated residue, such as Ser or Ala.

14. The cell-penetrating peptide (CPP) according to claim 13, wherein the residue corresponding to Cys-39 in SEQ ID NO: 2 is replaced with a serine.

15. The cell-penetrating peptide (CPP) according to claim 14 comprising SEQ ID NO: 16.

16. The cell-penetrating peptide (CPP) according to any one of the preceding claims, comprising an additional cysteine at its N- and/or C-terminus.

17. The cell-penetrating peptide (CPP) according to any one of the preceding claims comprising SEQ ID NO: 13, SEQ ID NO: 14 or SEQ ID NO:15.

18. A fusion protein comprising the cell-penetrating peptide (CPP) as defined in any one of claims 1 to 17 and a therapeutically useful protein.

19. The fusion protein according to claim 18 wherein the therapeutically useful protein is a dominant-negative protein; optionally wherein the dominant-negative protein is a dominant-negative mastermind-like protein (DN-MAML), AKT-DN or STAT3; further optionally wherein the DN-MAML comprises the sequence of SEQ ID NO: 9 or a sequence with 80% sequence identity thereto.

20. The fusion protein according to claim 20, wherein the cell-penetrating peptide (CPP) is covalently linked to the dominant-negative protein by a zero-length crosslinker, a homobifunctional crosslinker, a hetero-bifunctional crosslinker or a trifunctional crosslinkers;
optionally wherein
the zero-length crosslinker is selected from carbodiimides (EDC, EDC plus Sulfo-NHS, CMC, DCC, DIC), Woodward's Reagent K, N,N-Carbonyldiimidazole, Schiff Base Formation and Reductive Amination);
the homobifunctional crosslinkers is selected from Homobifunctional NHS Esters (DSP and DTSSP, DSS and BS3, DST and Sulfo-DST, BSOCOES and Sulfo-BSOCOES, EGS and Sulfo-EGS, DSG, DSC), Homobifunctional Imidoesters (DMA, DMP, DMS, DTBP), Homobifunctional Sulfhydryl-Reactive Crosslinkers (DPDPB, BMH), Difluorobenzene Derivatives (DFDNB, DFDNPS), Homobifunctional Photoreactive Crosslinkers (BASED), Homobifunctional Aldehydes (Formaldehyde, Glutaraldehyde), Bis-epoxides (1,4-Butanediol Diglycidyl Ether), Homobifunctional Hydrazides, Adipic Acid, Dihydrazide, Carbohydrazide), Bis-diazonium Derivatives (o-Tolidine, Diazotized, Bis-diazotized Benzidine), Bis-alkyl Halides;
the hetero-bifunctional Crosslinker is selected from Amine-Reactive and Sulfhydryl-Reactive Crosslinkers (SPDP, LC-SPDP, and Sulfo-LC-SPDP, SMPT and Sulfo-LC-SMPT, SMCC and Sulfo-SMCC, MBS and Sulfo-MBS, SIAB and Sulfo-SIAB, SMPB and Sulfo-SMPB, GMBS and Sulfo-GMBS, SIAX and SIAXX, SIAC and SIACX, NPIA), Carbonyl-Reactive and Sulfhydryl-Reactive Crosslinkers (MPBH, M2C2H, PDPH), Amine-Reactive and Photoreactive Crosslinkers (NHS-ASA, Sulfo-NHS-ASA, and Sulfo-NHS-LC-ASA, SASD, HSAB and Sulfo-HSAB, SANPAH and Sulfo-SANPAH, ANB-NOS, SAND, SADP and Sulfo-SADP, Sulfo-SAPB, SAED, Sulfo-SAMCA, p-Nitrophenyl Diazopyruvate, PNP-DTP), Sulfhydryl-Reactive and Photoreactive Crosslinkers (ASIB, APDP, Benzophenone-4-iodoacetamide, Benzophenone-4-maleimide), Carbonyl-Reactive and Photoreactive Crosslinkers (ABH), Carboxylate-Reactive and Photoreactive Crosslinkers (ASBA), Arginine-Reactive and Photoreactive Crosslinkers (APG); and
the trifunctional crosslinker is selected from 4-Azido-2-nitrophenylbiocytin-4-nitrophenyl ester, Sulfo-SBED, MTS-ATF-Biotin and MTS-ATF-LC-Biotin, Hydroxymethyl Phosphine Derivatives.

21. The fusion protein according to any one of claims 18 to 20, wherein
the cell-penetrating peptide (CPP) is as defined in claim 16;
the therapeutically useful protein is DN-MAML; and
DN-MAML is conjugated to the cell penetrating peptide via a thioester bond formed with a cysteine thiol at the N or C terminus of the ANTP sequence;
optionally wherein:
the DN-MAML protein comprises the sequence of SEQ ID NO: 9 or a sequence with 80% sequence identity thereto; and/or
the DN-MAML protein comprises a maleimide group.

22. A pharmaceutical composition comprising the cell penetrating peptide as defined in any one of claims 1 to 17 or the fusion protein as defined in any one of claims 18 to 21.

23. The cell penetrating peptide as defined in any one of claims 1 to 17 or the fusion protein as defined in any one of claims 18 to 21 or the pharmaceutical composition as defined in claim 22 for use in a method of therapy.

24. The cell penetrating peptide as defined in any one of claims 1 to 17 or the fusion protein as defined in any one of claims 18 to 21 or the pharmaceutical composition as defined in claim 22 for use in method of treating cancer.

25. The cell penetrating peptide, the fusion protein or the pharmaceutical composition for use as defined in claim 24, wherein administration of the cell penetrating peptide, fusion protein or the pharmaceutical composition inhibits Notch signalling in a cancer cell.

26. A method of treating cancer, the method comprising the administration of a cell penetrating peptide as defined in any one of claims 1 to 17 or the fusion protein as defined in any one of claims 18 to 21 or the pharmaceutical composition as defined in claim 22 to a subject in need thereof.

27. Use of the cell penetrating peptide as defined in any one of claims 1 to 17 or the fusion protein as defined in any one of claims 18 to 21 or the pharmaceutical composition as defined in claim 22 in the manufacture of a medicament for the treatment of cancer.

28. A method for producing a fusion protein, the method comprising linking the cell-penetrating peptide (CPP) as defined in any one of claims 1 to 17 and a therapeutically useful protein by any of the following methods: activated-esters coupling of lysine residues; methanesulfonyl acrylate coupling of lysine residues; copper and non-catalysed ‘click’ reactions of alkynes introduced into proteins; disulphide bonds formed with native or engineered cysteine thiol groups; thioether bonds formed with cysteine thiols and introduced maleimide groups; native chemical ligation; smartag chemo-selective ligation using formyl-glycine modified proteins; chemoselective azo-coupling reactions; cyclo-addition reactions (Cu-AAC or SPAAC) of azide-derivatized amino acids; and/or photo-reactive or photo-catalysed chemical reactions.

29. A method for producing an ANTP fusion protein, the method comprising modifying the ANTP sequence by site directed mutagenesis to (i) replace the residue corresponding to Cys-39 in SEQ ID NO: 2 with a serine, and (ii) add a cysteine to the N or C terminus of the ANTP sequence, and conjugating the modified ANTP sequence to a therapeutic protein using thiol-maleimide chemistry.

30. The method of claim 29, wherein the therapeutic protein is DN-MAML or a derivative thereof.

31. The method of claim 30, wherein the DN-MAML has the sequence of SEQ ID NO: 9 or a sequence with 80% sequence identity thereto.

32. The method of claim 30 or 31, wherein the DN-MAML sequence has bene modified to introduce a maleimide group.

33. The method of any one of claims 29 to 32, wherein the ANTP sequence comprises SEQ ID NO: 2 or a sequence having 80% sequence identity thereto.


SEQUENCE LISTING (19 sequences)

SEQ ID NO: 1 - protein, 60 aa, Artificial Sequence
  Homeodomain consensus sequence
RRRKRTAYTRYQLLELEKEFLFNRYLTRRRRIELAHSLNLTERHIKIWFQNRRMKWKKEN

SEQ ID NO: 2 - protein, 60 aa, Artificial Sequence
  Amino acid sequence of the Antennapedia homeodomain
RKRGRQTYTRYQTLELEKEFHFNRYLTRRRRIEIAHALCLTERQIKIWFQNRRMKWKKEN

SEQ ID NO: 3 - protein, 60 aa, Artificial Sequence
  Amino acid sequence of the Antennapedia homeodomain with conservative substitutions
  MISC_FEATURE (1)..(38): Wherein X is K or R
  MISC_FEATURE (39)..(39): Wherein X is A, C, G or S
  MISC_FEATURE (40)..(60): Wherein X is R or K
XXXGXQTYTXYQTLELEXEFHFNXYLTXXXXIEIAHALXLTEXQIXIWFQNXXMXWXXEN

SEQ ID NO: 4 - protein, 16 aa, Artificial Sequence
  Penetratin
RQIKIWFQNRRMKWKK

SEQ ID NO: 5 - protein, 16 aa, Artificial Sequence
  penetratin variant
  MISC_FEATURE (1)..(16): Wherein X is R or K
XQIXIWFQNXXMXWXX

SEQ ID NO: 6 - protein, 1016 aa, Homo sapiens
MVLPTCPMAEFALPRHSAVMERLRRRIELCRRHHSTCEARYEAVSPERLELERQHTFALHQRCIQAKAKRAGKHRQPPAATAPAPAAPAPRLDAADGPEHGRPATHLHDTVKRNLDSATSPQNGDQQNGYGDLFPGHKKTRREAPLGVAISSNGLPPASPLGQSDKPSGADALQSSGKHSLGLDSLNKKRLADSSLHLNGGSNPSESFPLSLNKELKQEPVEDLPCMITGTVGSISQSNLMPDLNLNEQEWKELIEELNRSVPDEDMKDLFNEDFEEKKDPESSGSATQTPLAQDINIKTEFSPAAFEQEQLGSPQVRAGSAGQTFLGPSSAPVSTDSPSLGGSQTLFHTSGQPRADNPSPNLMPASAQAQNAQRALAGVVLPSQGPGGASELSSAHQLQQIAAKQKREQMLQNPQQATPAPAPGQMSTWQQTGPSHSSLDVPYPMEKPASPSSYKQDFTNSKLLMMPSVNKSSPRPGGPYLQPSHVNLLSHQPPSNLNQNSANNQGSVLDYGNTKPLSHYKADCGQGSPGSGQSKPALMAYLPQQLSHISHEQNSLFLMKPKPGNMPFRSLVPPGQEQNPSSVPVQAQATSVGTQPPAVSVASSHNSSPYLSSQQQAAVMKQHQLLLDQQKQREQQQKHLQQQQFLQRQQHLLAEQEKQQFQRHLTRPPPQYQDPTQGSFPQQVGQFTGSSAAVPGMNTLGPSNSSCPRVFPQAGNLMPMGPGHASVSSLPTNSGQQDRGVAQFPGSQNMPQSSLYGMASGITQIVAQPPPQATNGHAHIPRQTNVGQNTSVSAAYGQNSLGSSGLSQQHNKGTLNPGLTKPPVPRVSPAMGGQNSSWQHQGMPNLSGQTPGNSNVSPFTAASSFHMQQQAHLKMSSPQFSQAVPNRPMAPMSSAAAVGSLLPPVSAQQRTSAPAPAPPPTAPQQGLPGLSPAGPELGAFSQSPASQMGGRAGLHCTQAYPVRTAGQELPFAYSGQPGGSGLSSVAGHTDLIDSLLKNRTSEEWMSDLDDLLGSQ

SEQ ID NO: 7 - protein, 1096 aa, Homo sapiens
GRAESSDRERESTLQLLSLVQHGQGARKAGKHTKATATAATTTAPPPPPAAPPAASQAAATAAPPPPPDYHHHHQQHLLNSSNNGGSGGINGEQQPPASTPGDQRNSALIALQGSLKRKQVVNLSPANSKRPNGFVDNSFLDIKRIRVGENLSAGQGGLQINNGQSQIMSGTLPMSQAPLRKTNTLPSHTHSPGNGLFNMGLKEVKKEPGETLSCSKHMDGQMTQENIFPNRYGDDPGEQLMDPELQELFNELTNISVPPMSDLELENMINATIKQDDPFNIDLGQQSQRSTPRPSLPMEKIVIKSEYSPGLTQGPSGSPQLRPPSAGPAFSMANSALSTSSPIPSVPQSQAQPQTGSGASRALPSWQEVSHAQQLKQIAANRQQHARMQQHQQQHQPTNWSALPSSAGPSPGPFGQEKIPSPSFGQQTFSPQSSPMPGVAGGSGQSKVMANYMYKAGPSAQGGHLDVLMQQKPQDLSRSFINNPHPAMEPRQGNTKPLFHFNSDQANQQMPSVLPSQNKPSLLHYTQQQQQQQQQQQQQQQQQQQQQQQQQQQQQQQQQQSSISAQQQQQQQSSISAQQQQQQQQQQQQQQQQQQQQQQQQQQQPSSQPAQSLPSQPLLRSPLPLQQKLLLQQMQNQPIAGMGYQVSQQQRQDQHSVVGQNTGPSPSPNPCSNPNTGSGYMNSQQSLLNQQLMGKKQTLQRQIMEQKQQLLLQQQMLADAEKIAPQDQINRHLSRPPPDYKDQRRNVGNMQPTAQYSGGSSTISLNSNQALANPVSTHTILTPNSSLLSTSHGTRMPSLSTAVQNMGMYGNLPCNQPNTYSVTSGMNQLTQQRNPKQLLANQNNPMMPRPPTLGPSNNNNVATFGAGSVGNSQQLRPNLTHSMASMPPQRTSNVMITSNTTAPNWASQEGTSKQQEALTSAGVRFPTGTPAAYTPNQSLQQAVGSQQFSQRAVAPPNQLTPAVQMRPMNQMSQTLNGQTMGPLRGLNLRPNQLSTQILPNLNQSGTGLNQSRTGINQPPSLTPSNFPSPNQSSRAFQGTDHSSDLAFDFLSQQNDNMGPALNSDADFIDSLLKTEPGNDDWMKDINLDEILGNNS

SEQ ID NO: 8 - protein, 1133 aa, Homo sapiens
MGDFAAPAAAANGSSICINSSLNSSLGGAGIGVNNTPNSTPAAPSSNHPAAGGCGGSGGPGGGSAAVPKHSTVVERLRQRIEGCRRHHVNCENRYQQAQVEQLELERRDTVSLYQRTLEQRAKKSGAGTGKQQHPSKPQQDAEAASAEQRNHTLIMLQETVKRKLEGARSPLNGDQQNGACDGNFSPTSKRIRKDISAGMEAINNLPSNMPLPSASPLHQLDLKPSLPLQNSGTHTPGLLEDLSKNGRLPEIKLPVNGCSDLEDSFTILQSKDLKQEPLDDPTCIDTSETSLSNQNKLFSDINLNDQEWQELIDELANTVPEDDIQDLFNEDFEEKKEPEFSQPATETPLSQESASVKSDPSHSPFAHVSMGSPQARPSSSGPPFSTVSTATSLPSVASTPAAPNPASSPANCAVQSPQTPNQAHTPGQAPPRPGNGYLLNPAAVTVAGSASGPVAVPSSDMSPAEQLKQMAAQQQQRAKLMQQKQQQQQQQQQQQQQQQQQQQQQHSNQTSNWSPLGPPSSPYGAAFTAEKPNSPMMYPQAFNNQNPIVPPMANNLQKTTMNNYLPQNHMNMINQQPNNLGTNSLNKQHNILTYGNTKPLTHFNADLSQRMTPPVANPNKNPLMPYIQQQQQQQQQQQQQQQQQQPPPPQLQAPRAHLSEDQKRLLLMKQKGVMNQPMAYAALPSHGQEQHPVGLPRTTGPMQSSVPPGSGGMVSGASPAGPGFLGSQPQAAIMKQMLIDQRAQLIEQQKQQFLREQRQQQQQQQQILAEQQLQQSHLPRQHLQPQRNPYPVQQVNQFQGSPQDIAAVRSQAALQSMRTSRLMAQNAGMMGIGPSQNPGTMATAAAQSEMGLAPYSTTPTSQPGMYNMSTGMTQMLQHPNQSGMSITHNQAQGPRQPASGQGVGMVSGFGQSMLVNSAITQQHPQMKGPVGQALPRPQAPPRLQSLMGTVQQGAQSWQQRSLQGMPGRTSGELGPFNNGASYPLQAGQPRLTKQHFPQGLSQSVVDANTGTVRTLNPAAMGRQMMPSLPGQQGTSQARPMVMSGLSQGVPGMPAFSQPPAQQQIPSGSFAPSSQSQAYERNAPQDVSYNYSGDGAGGSFPGLPDGADLVDSIIKGGPGDEWMQELDELFGNP

SEQ ID NO: 9 - protein, 62 aa, Artificial Sequence
  dnMAML(13-74)
LPRHSAVMERLRRRIELCRRHHSTCEARYEAVSPERLELERQHTFALHQRCIQAKAKRAGKH

SEQ ID NO: 10 - protein, 127 aa, Artificial Sequence
  ANTP-dnMAML conjugate
RKRGRQTYTRYQTLELEKEFHFNRYLTRRRRIEIAHALCLTERQIKIWFQNRRMKWKKENGEFMALPRHSAVMERLRRRIELCRRHHSTCEARYEAVSPERLELERQHTFALHQRCIQAKAKRAGKH

SEQ ID NO: 11 - protein, 85 aa, Artificial Sequence
  penetratin-dnMAML conjugate
RQIKIWFQNRRMKWKKENGEFMALPRHSAVMERLRRRIELCRRHHSTCEARYEAVSPERLELERQHTFALHQRCIQAKAKRAGKH

SEQ ID NO: 12 - protein, 136 aa, Artificial Sequence
  His-ANTP-dnMAML conjugate (Syntana-4)
MHHHHHHGSRKRGRQTYTRYQTLELEKEFHFNRYLTRRRRIEIAHALCLTERQIKIWFQNRRMKWKKENGEFMALPRHSAVMERLRRRIELCRRHHSTCEARYEAVSPERLELERQHTFALHQRCIQAKAKRAGKH

SEQ ID NO: 13 - protein, 61 aa, Artificial Sequence
  Amino acid sequence of ANTP variant
RKRGRQTYTRYQTLELEKEFHFNRYLTRRRRIEIAHALSLTERQIKIWFQNRRMKWKKENC

SEQ ID NO: 14 - protein, 61 aa, Artificial Sequence
  Amino acid sequence of ANTP variant
CRKRGRQTYTRYQTLELEKEFHFNRYLTRRRRIEIAHALSLTERQIKIWFQNRRMKWKKEN

SEQ ID NO: 15 - protein, 62 aa, Artificial Sequence
  Amino acid sequence of ANTP variant
CRKRGRQTYTRYQTLELEKEFHFNRYLTRRRRIEIAHALSLTERQIKIWFQNRRMKWKKENC

SEQ ID NO: 16 - protein, 60 aa, Artificial Sequence
  ANTP variant
RKRGRQTYTRYQTLELEKEFHFNRYLTRRRRIEIAHALSLTERQIKIWFQNRRMKWKKEN

SEQ ID NO: 17 - protein, 18 aa, Artificial Sequence
  amino acid sequence of an amphipathic model peptide
KLALKLALKALKAALKLA

SEQ ID NO: 18 - protein, 5 aa, Artificial Sequence
  Exemplary flexible linker
GGGGS

SEQ ID NO: 19 - protein, 12 aa, Artificial Sequence
  Gly and Ser rich flexible linker
GSAGSAAGSGEF